(19) United States
(12) Patent Application Publication
(10) Pub. No.: US 2012/0238762 A1
(43) Pub. Date: Sep. 20, 2012

(54) AZOLE DERIVATIVES AND METHODS FOR PRODUCING THE SAME, INTERMEDIATE COMPOUNDS FOR THE DERIVATIVES AND METHODS FOR PRODUCING THE SAME, AND AGRO-HORTICULTURAL AGENTS AND INDUSTRIAL MATERIAL PROTECTING AGENTS CONTAINING THE DERIVATIVES

(75) Inventors: Keiichi Sudo, Tokyo (JP); Takashi Shimokawara, Tokyo (JP); Eiyu Imai, Tokyo (JP); Nobuyuki Kusano, Tokyo (JP); Hisashi Kanno, Tokyo (JP); Taiji Miyake, Tokyo (JP); Masaru Mori, Tokyo (JP); Toshihide Saishoji, Tokyo (JP)

(73) Assignee: KUREHA CORPORATION, Tokyo (JP)

(21) Appl. No.: 13/508,337

(22) PCT Filed: Nov. 29, 2010
(86) PCT No.: PCT/JP2010/006948

§ 371 Date: May 30, 2012

(30) Foreign Application Priority Data
Dec. 8, 2009 (JP) 2009-278850

(51) Int. Cl. C07D 249/08; C07D 301/02; C07D 303/22; C07D 301/00; C07D 303/04; C07D 303/08

(52) U.S. Cl. 548/268.2; 549/512; 549/563; 549/554; 549/513; 549/519

(57) ABSTRACT

To provide an azole derivative which is contained as an active ingredient in an agro-horticultural agent having an excellent controlling effect on diseases. An azole derivative according to the invention is represented by Formula (I), wherein R¹ and R² are same or different, and each denotes a substituted C3-C6 cycloalkyl group or a C1-C4 alkyl group substituted with the substituted C3-C6 cycloalkyl group; and A denotes a nitrogen atom or a methyne group.

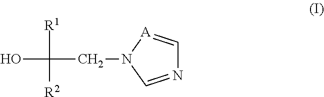

### TECHNICAL FIELD

The present invention relates to a novel azole derivative. It also relates to an agro-horticultural agent and an industrial material protecting agent containing the derivative as an active ingredient and a method for producing the derivative.

### BACKGROUND ART

Conventionally, a large number of hydroxyethylazole derivatives, each being a 5-membered heterocyclic ring containing one or more nitrogen atoms in the ring which is a derivative whose hydroxyl group-carrying carbon atom is further bound to a cycloalkyl group or a cycloalkyl group-substituted alkyl group, are proposed as active ingredients of agro-horticultural biocides (see, for example, Patent Literatures 1 to 13).

### CITATION LIST

### Patent Literature

[PTL 1] European Patent Application Publication No. 0015756 Specification
[PTL 2] European Patent Application Publication No. 0052424 Specification
[PTL 3] European Patent Application Publication No. 0061835 Specification
[PTL 4] European Patent Application Publication No. 0297345 Specification
[PTL 5] European Patent Application Publication No. 0047594 Specification
[PTL 6] European Patent Application Publication No. 0212605 Specification
[PTL 7] Japanese Unexamined Patent Application Publication No. 56-97276
[PTL 8] Japanese Unexamined Patent Application Publication No. 61-126049
[PTL 9] Japanese Unexamined Patent Application Publication No. 2-286664
[PTL 10] Japanese Unexamined Patent Application Publication No. 59-98061
[PTL 11] Japanese Unexamined Patent Application Publication No. 61-271276
[PTL 12] European Patent Application Publication No. 0229642 Specification
[PTL 13] Japanese Unexamined Patent Application Publication No. 4-230270

### SUMMARY OF INVENTION

### Technical Problem

Conventionally, an agro-horticultural pesticide having a low toxicity to humans, capable of being handled safely, and exhibiting a high controlling effect on a wide range of plant diseases has been desired. Also, there has been a need for a plant growth regulator which regulates the growth of a variety of crops and horticultural plants thereby exhibiting yield-increasing effects or quality-improving effects, as well as an industrial material protecting agent which protects an industrial material from a wide range of hazardous microorganisms which invade such materials.

Accordingly, the present invention aims primarily at providing an azole derivative contained as an active ingredient in an agro-horticultural agent and an industrial material which fulfill the need described above.

### Solution to Problem

To achieve the aim mentioned above, we made an extensive study on chemical structures and biological activities of a large number of azole derivatives. As a result, we found that an azole derivative represented by Formula (I) shown below has an excellent activity, thus establishing the present invention.

Thus, the invention is based on such novel findings, and includes the following inventive aspects.

An azole derivative according to the invention is represented by Formula (I):

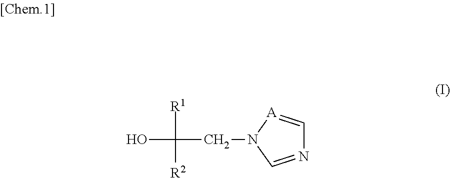

wherein R¹ and R² are same or different, and each denotes a C3-C6 cycloalkyl group or a C1-C4 alkyl group substituted with the cycloalkyl group;

the cycloalkyl group and the alkyl group may be substituted with a halogen atom, a C1-C4 alkyl group, a C1-C4 haloalkyl group, a C3-C6 cycloalkyl group, an aryl group, or an arylalkyl group (alkyl moiety carbon chain being C1-C3);

the aromatic ring of the aryl group and the arylalkyl group may be substituted with a halogen atom, a C1-C4 alkyl group, a C1-C4 haloalkyl group, a C1-C4 alkoxy group, or a C1-C4 haloalkoxy group; and,

A denotes a nitrogen atom or a methyne group.

The azole derivative having the structure shown above is advantageous in that it has an excellent biocidal effect on a large number of microorganisms which induce diseases in plants.

In the azole derivatives according to the invention, it is preferred that each of R¹ and R² in Formula (I) described above is a C3-C6 cycloalkyl group substituted with a halogen atom, a C1-C4 alkyl group, or a C1-C4 haloalkyl group, or a C1-C4 alkyl group substituted with the substituted C3-C6 cycloalkyl group.

In the azole derivatives according to the invention, it is further preferred that each of R¹ and R² in Formula (I) described above is a cyclopropyl group substituted with a halogen atom or a C1-C4 alkyl group, or a C1-C4 alkyl group substituted with the substituted cyclopropyl group.

In the azole derivatives according to the invention, it is preferred that each of R¹ and R² in Formula (I) described above is represented by Formula (XVII):

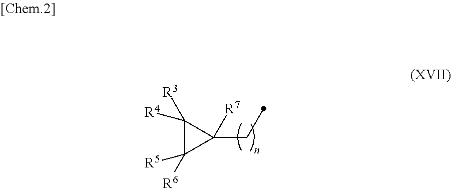

wherein each of R³, R⁴, R⁵, R⁶, and R⁷ denotes a hydrogen atom, a halogen atom, a methyl group or an ethyl group, and at least one of R³, R⁴, R⁵, R⁶, and R⁷ denotes a halogen atom, and n denotes 0 to 2.

Herein, the carbon marked with a dot in Formula (XVII) described above represents the carbon atom identical to the carbon atom having a hydroxyl group in Formula (I).

In the azole derivatives according to the invention, it is preferred that, when n in Formula (XVII) described above representing R¹ is 1 to 2, then n in Formula (XVII) described above representing R² is 0 while R⁷ is a halogen atom and each of R³, R⁴, R⁵, and R⁶ is a hydrogen atom.

In addition, in the azole derivatives according to the invention, it is preferred that A in Formula (I) described above is a nitrogen atom.

As a result of having the structure mentioned above, the azole derivative according to the invention is advantageous in that it has a further excellent biocidal effect on a large number of microorganisms which induce diseases in plants.

The invention also includes the following compounds as intermediates for the azole derivatives described above.

That is, the intermediates for the azole derivatives according to the invention are oxirane compounds represented by Formula (II):

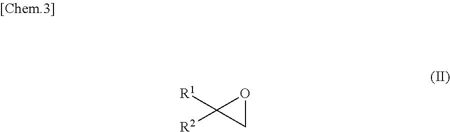

wherein R¹ and R² are same or different, and each denotes a C3-C6 cycloalkyl group, a C1-C4 alkyl group substituted with the cycloalkyl group, a C2 alkenyl group, or a C1-C4 alkyl group substituted with the alkenyl group; the cycloalkyl group, the alkyl group, or the alkenyl group may be substituted with a halogen atom, a C1-C4 alkyl group, a C1-C4 haloalkyl group, a C3-C6 cycloalkyl group, an aryl group, or an arylalkyl group (alkyl moiety carbon chain being C1-C3); the aromatic ring of the aryl group and the arylalkyl group may be substituted with a halogen atom, a C1-C4 alkyl group, a C1-C4 haloalkyl group, a C1-C4 alkoxy group, or a C1-C4 haloalkoxy group.

The intermediate compounds for the azole derivatives according to the invention are preferably the oxirane compounds represented by Formula (II-a):

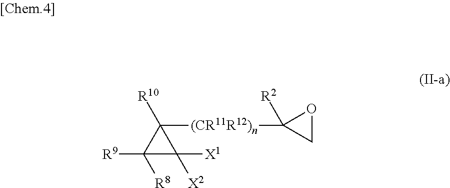

wherein R⁸, R⁹, R¹⁰, R¹¹, and R¹² may be substituted with a hydrogen atom, a halogen atom, a C1-C4 alkyl group, a C1-C4 haloalkyl group, a C3-C6 cycloalkyl group, an aryl group, or an arylalkyl group (alkyl moiety carbon chain being C1-C3); the aromatic ring of the aryl group and the arylalkyl group may be substituted with a halogen atom, a C1-C4 alkyl group, a C1-C4 haloalkyl group, a C1-C4 alkoxy group, or a C1-C4 haloalkoxy group; each of X¹ and X² denotes a halogen atom; and n denotes 0 to 4.

The intermediate compounds for the azole derivatives according to the invention are preferably the oxirane compounds represented by Formula (VIII):

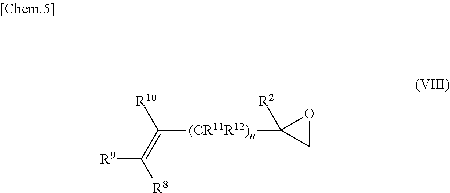

wherein each of R⁸, R⁹, R¹⁰, R¹¹, and R¹² denotes a hydrogen atom, a halogen atom, a C1-C4 alkyl group, a C1-C4 haloalkyl group, a C3-C6 cycloalkyl group, an aryl group, or an arylalkyl group (alkyl moiety carbon chain being C1-C3); the aromatic ring of the aryl group and the arylalkyl group may be substituted with a halogen atom, a C1-C4 alkyl group, a C1-C4 haloalkyl group, a C1-C4 alkoxy group, or a C1-C4 haloalkoxy group; and n denotes 0 to 4.

Moreover, a method for producing the azole derivative according to the invention comprises a step of reacting an oxirane compound represented by Formula (II):

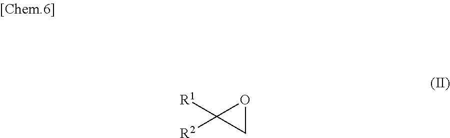

with a 1,2,4-triazole or imidazole compound represented by Formula (III):

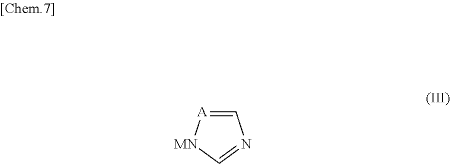

wherein M denotes a hydrogen atom or an alkaline metal; and A denotes a nitrogen atom or a methyne group.

The invention also includes the following methods as methods for producing the intermediates for the azole derivatives described above.

The methods for producing the intermediate compounds for the azole derivatives according to the invention comprise a step of subjecting an oxirane compound represented by Formula (VIII) to conversion into a gem-dihalocyclopropane thereby obtaining an intermediate compound represented by Formula (II-a).

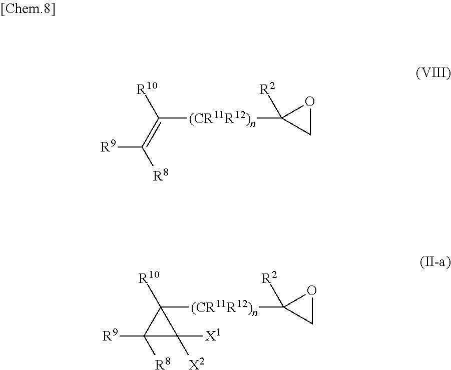

Moreover, the methods for producing the intermediate compounds for the azole derivatives according to the invention comprise a step of allowing a compound represented by Formula (VII) to react with an organometallic compound represented by Formula (X), to obtain a halohydrin compound represented by Formula (IX) which is then subjected to conversion into an oxirane, thereby obtaining an intermediate compound represented by Formula (VIII):

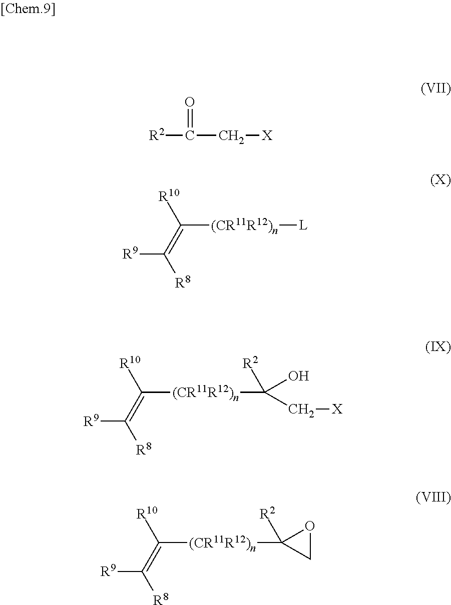

wherein L in Formula (X) denotes an alkaline metal, an alkaline earth metal-Q₁ (Q₁ is an halogen atom), a ½ (Cu alkaline metal), a zinc-Q₂ (Q₂ is a halogen atom), and X in Formulae (VII) and (IX) denotes a halogen atom.

Furthermore, the methods for producing the intermediate compounds for the azole derivatives according to the invention comprise a step of subjecting a carbonyl compound represented by Formula (XI) to conversion into an oxirane thereby obtaining an intermediate compound represented by Formula (VIII-a):

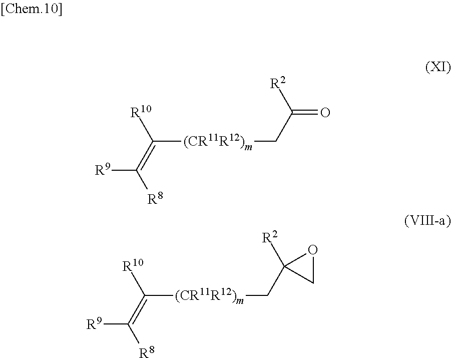

wherein m in Formula (XI) and (VIII-a) denotes 1 to 3.

Also included in the invention is an agro-horticultural agent or an industrial material protecting agent containing an azole derivative according to the invention as an active ingredient.

In the specification and related matters, a symbol defining an identical functional group (or atom) in each formula is indicated with the identical symbol while omitting its detailed description. For example, an R² shown in Formula (I) and an R² shown in a different formula are identical. This understanding is not limited to R², and is also applicable to other functional groups (or atoms).

### Advantageous Effects of Invention

An azole derivative according to the invention has an excellent biocidal effect on a large number of microorganisms which induce diseases in plants. Therefore, an agro-horticultural agent containing the azole derivative according to the invention as an active ingredient can advantageously exhibit a high controlling effect on a wide range of plant diseases. Moreover, the agro-horticultural agent containing the azole derivative according to the invention as an active ingredient can advantageously regulate the growth of a variety of crops and horticultural plants thereby increasing their yields while improving their qualities. On the other hand, an industrial material protecting agent containing the azole derivative according to the invention as an active ingredient can further advantageously protect an industrial material from a wide range of hazardous microorganisms which invade such materials.

### DESCRIPTION OF EMBODIMENTS

The embodiments in the best mode for carrying out the invention are described below. These embodiments are just examples of the representative embodiments of the invention and do not serve to allow the scope of the invention to be interpreted narrowly. The descriptions are made in the following orders.

In the embodiments, an identical term is used to refer to an identical meaning unless otherwise specified. This understanding is also applicable to a substituent or an atom in a formula as well as to a symbol indicating the number thereof.
1. Azole derivatives

### (1) R¹ and R²

### (2) A

### (3) Isomers

(4) Typical examples
2. Methods for producing azole derivatives

### (1) Solvents

(2) Bases and acids
(3) First method for producing Compound (I)

### (3-1) Step A1

### (3-2) Step A2

### (3-3) Step A3

### (3-4) Step A2a

### (3-5) Step A4

### (3-6) Step A4a

(4) Second method for producing Compound (I)

### (4-1) Step B1

### (4-2) Step B2

3. Agro-horticultural agents and industrial material protecting agents
(1) Plant disease controlling effects
(2) Plant growth promoting effect
(3) Industrial material protecting effect

### (4) Formulations

### 1. Azole Derivatives

An azole derivative represented by Formula (I) described above according to the invention (hereinafter referred to as Compound (I)) is described below.

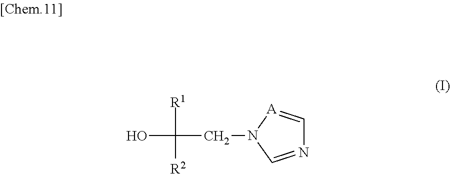

The contexts of the definitions of respective symbols (R¹, R², A) in Compound (I) and their typical examples are described below.

(1) R¹ and R²

Each of R¹ and R² denotes a C3-C6 cycloalkyl group or a C1-C4 alkyl group substituted with a C3-C6 cycloalkyl group. R¹ and R² may be same or different.

The C3-C6 cycloalkyl group may be, for example, a cycropropyl group, a cyclobutyl group, a cyclopentyl group, a cyclohexyl group, and the like, and is more preferably a cycropropyl group, a cyclobutyl group, and a cyclopentyl group, and especially preferably a cycropropyl group. The C1-C4 alkyl group substituted with a C3-C6 cycloalkyl group may be, for example, a cyclopropylmethyl group, a cyclobutylmethyl group, a 2-(cyclopropyl)ethyl, a cyclopentylmethyl group, a cyclohexylmethyl group, a 3-(cyclopropyl)propyl group, a 4-(cyclopropyl)butyl group, and the like, and is more preferably a cyclopropylmethyl group, a 2-(cyclopropyl)ethyl, a 3-(cyclopropyl)propyl group, and a 4-(cyclopropyl)butyl group, and especially preferably a cyclopropylmethyl group and a 2-(cyclopropyl)ethyl.

These groups may be substituted with a halogen atom, a C1-C4 alkyl group, a C1-C4 haloalkyl group, a C3-C6 cycloalkyl group, an aryl group, or an arylalkyl group (alkyl moiety carbon chain being C1-C3).

The halogen atom may be, for example, a fluorine atom, a chlorine atom, a bromine atom, and an iodine atom. The C1-C4 alkyl group may be, for example, a methyl group, an ethyl group, a n-propyl group, and an isopropyl group. The C1-C4 haloalkyl group may be, for example, a trifluoromethyl group, a 1,1,2,2,2-pentafluoroethyl group, a chloromethyl group, a trichloromethyl group, and a bromomethyl group. The C3-C6 cycloalkyl group may be, for example, a cyclopropyl group and a cyclobutyl group. The aryl group may be, for example, a phenyl group. The arylalkyl group may be, for example, a benzyl group and a phenethyl group.

Among these, those further preferred may be, for example, a fluorine atom, a chlorine atom, a bromine atom, and an iodine atom as halogen atoms, and a methyl group, an ethyl group, and an n-propyl group as C1-C4 alkyl groups. The C1-C4 haloalkyl group may be, for example, a trifluoromethyl group, a chloromethyl group, and a trichloromethyl group. The C3-C6 cycloalkyl group may be, for example, a cyclopropyl group. The aryl group may be, for example, a phenyl group.

More preferable substituents may be, for example, a fluorine atom, a chlorine atom, a bromine atom, a methyl group, an ethyl group, a cyclopropyl group, and a phenyl group.

Those especially preferred may be, for example, a chlorine atom, a bromine atom, and a methyl group.

The phenyl moiety of the aryl group and the arylalkyl group described above may be mono- to tri-substituted with a halogen atom, a C1-C4 alkyl group, a C1-C4 haloalkyl group, a C1-C4 alkoxy group, or a C1-C4 haloalkoxy group.

The substituent which substitute the phenyl moiety of these aryl group and arylalkyl group may be exemplified below.

The halogen atom may be, for example, a fluorine atom, a chlorine atom, a bromine atom and an iodine atom. The C1-C4 alkyl group may be, for example, a methyl group, an ethyl group, an n-propyl group, an isopropyl group, an n-butyl group, an isobutyl group, a sec-butyl group, a tert-butyl group, and a cyclopropylmethyl group. The C1-C4 haloalkyl group may be, for example, a trifluoromethyl group, a 1,1,2,2,2-pentafluoroethyl group, a chloromethyl group, a trichloromethyl group, and a bromomethyl group. The C1-C4 alkoxy group may be, for example, a methoxy group, an ethoxy group, an isopropoxy group, and a tert-butoxy group. The C1-C4 haloalkoxy group may be, for example, a trifluoromethoxy group, a 2,2,2-trifluoroethoxy group, and a 1,1,2,2,2-pentafluoroethoxy group.

Those which may be more preferably exemplified are a fluorine atom, a chlorine atom, a bromine atom, a methyl group, an ethyl group, a trifluoromethyl group, a chloromethyl group, a methoxy group, and an ethoxy group.

(2) A

A denotes a nitrogen atom or a methyne group.

(3) Isomers

In Compound (I) wherein R¹ and R² are different, the carbon atom to which a hydroxyl group is bound becomes an asymmetric carbon atom. Also depending on the structure represented by R¹ and R², an asymmetric carbon atom occurs. Accordingly, Compound (I) may exist as geometric isomers and optical isomers. It should be understood that Compound (I) includes all individual isomers and any mixtures of respective isomers in any ratio.

(4) Typical examples

Depending on the combination of R¹, R², and A described above, the compounds indicated in Table 1 to Table 37 shown below can be exemplified as Compounds (I).

TABLE 1

| Compound No. | R1 | R2 | A |
|---|---|---|---|
| I-1 | 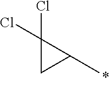 | 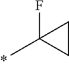 | N |
| I-2 | | 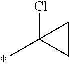 | N |
| I-3 | | 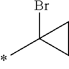 | N |
| I-4 | | 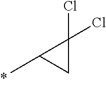 | N |
| I-5 | | 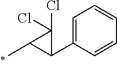 | N |
| I-6 | 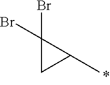 | 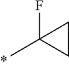 | N |
| I-7 | | 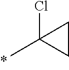 | N |
| I-8 | | 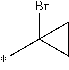 | N |
| I-9 | | 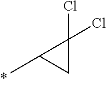 | N |
| I-10 | | 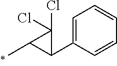 | N |
| I-11 | 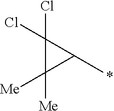 | 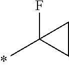 | N |
| I-12 | | 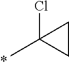 | N |
| I-13 | | 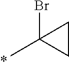 | N |
| I-14 | | 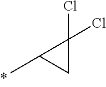 | N |
| I-15 | | 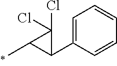 | N |
| I-16 | 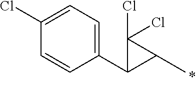 | 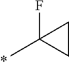 | N |
| I-17 | | 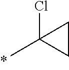 | N |
| I-18 | | 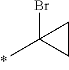 | N |
| I-19 | | 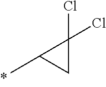 | N |
| I-20 | | 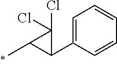 | N |

TABLE 2

| Compound No. | R1 | R2 | A |
|---|---|---|---|
| I-21 | 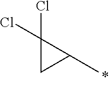 | 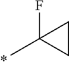 | CH |
| I-22 | | 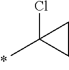 | CH |
| I-23 | | 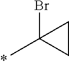 | CH |
| I-24 | | 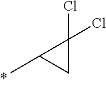 | CH |
| I-25 | | 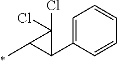 | CH |
| I-26 | 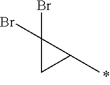 | 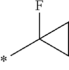 | CH |
| I-27 | | 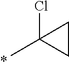 | CH |
| I-28 | | 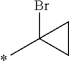 | CH |
| I-29 | | 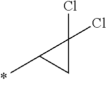 | CH |
| I-30 | | 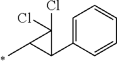 | CH |
| I-31 | 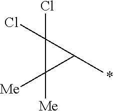 | 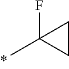 | CH |
| I-32 | | 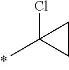 | CH |
| I-33 | | 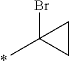 | CH |
| I-34 | | 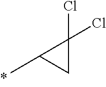 | CH |
| I-35 | | 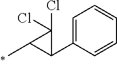 | CH |
| I-36 | 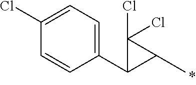 | 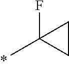 | CH |
| I-37 | | 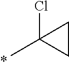 | CH |
| I-38 | | 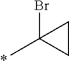 | CH |
| I-39 | | 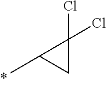 | CH |
| I-40 | | 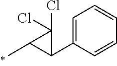 | CH |

TABLE 3

| Compound No. | R1 | R2 | A |
|---|---|---|---|
| I-41 | 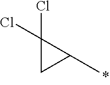 | 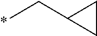 | N |
| I-42 | | 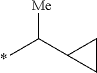 | N |
| I-43 | | 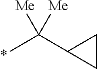 | N |
| I-44 | | 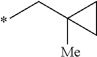 | N |
| I-45 | | 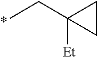 | N |
| I-46 | | 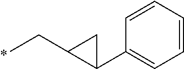 | N |
| I-47 | | 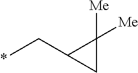 | N |
| I-48 | | 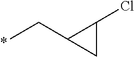 | N |
| I-49 | | 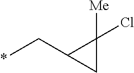 | N |
| I-50 | | 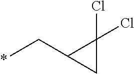 | N |
| I-51 | | 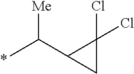 | N |
| I-52 | | 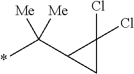 | N |
| I-53 | | 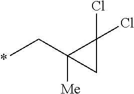 | N |
| I-54 | | 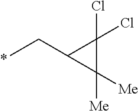 | N |
| I-55 | | 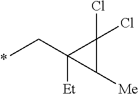 | N |
| I-56 | | 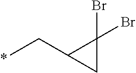 | N |

TABLE 4

| Compound No. | R1 | R2 | A |
|---|---|---|---|
| I-57 | 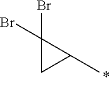 | 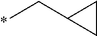 | N |
| I-58 | | 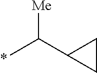 | N |
| I-59 | | 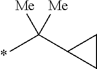 | N |
| I-60 | | 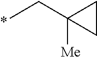 | N |
| I-61 | | 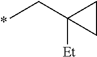 | N |
| I-62 | | 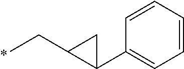 | N |
| I-63 | | 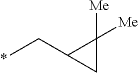 | N |
| I-64 | | 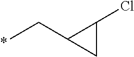 | N |
| I-65 | | 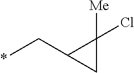 | N |
| I-66 | | 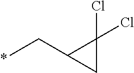 | N |
| I-67 | | 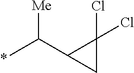 | N |
| I-68 | | 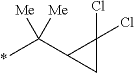 | N |
| I-69 | | 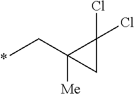 | N |
| I-70 | | 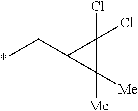 | N |
| I-71 | | 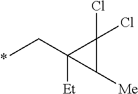 | N |
| I-72 | | 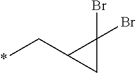 | N |

TABLE 5

| Compound No. | R1 | R2 | A |
|---|---|---|---|
| I-73 | 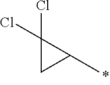 | 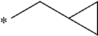 | CH |
| I-74 | | 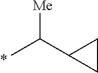 | CH |
| I-75 | | 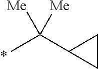 | CH |
| I-76 | | 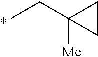 | CH |
| I-77 | | 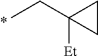 | CH |
| I-78 | | 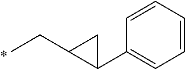 | CH |
| I-79 | | 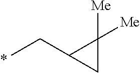 | CH |
| I-80 | | 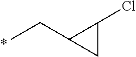 | CH |
| I-81 | | 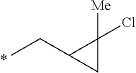 | CH |
| I-82 | | 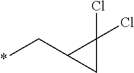 | CH |
| I-83 | | 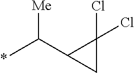 | CH |
| I-84 | | 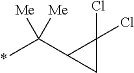 | CH |
| I-85 | | 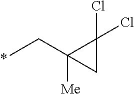 | CH |
| I-86 | | 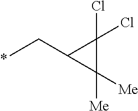 | CH |
| I-87 | | 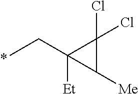 | CH |
| I-88 | | 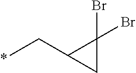 | CH |

TABLE 6

| Compound No. | R1 | R2 | A |
|---|---|---|---|
| I-89 | 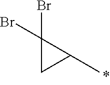 | 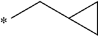 | CH |
| I-90 | | 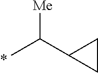 | CH |
| I-91 | | 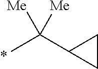 | CH |
| I-92 | | 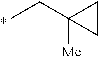 | CH |
| I-93 | | 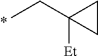 | CH |
| I-94 | | 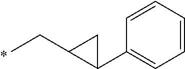 | CH |
| I-95 | | 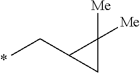 | CH |
| I-96 | | 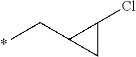 | CH |
| I-97 | | 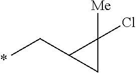 | CH |
| I-98 | | 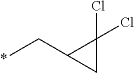 | CH |
| I-99 | | 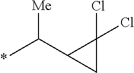 | CH |
| I-100 | | 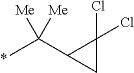 | CH |
| I-101 | | 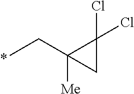 | CH |
| I-102 | | 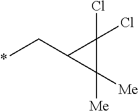 | CH |
| I-103 | | 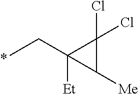 | CH |
| I-104 | | 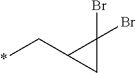 | CH |

TABLE 7

| Compound No. | R1 | R2 | A |
|---|---|---|---|
| I-105 | 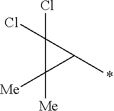 | 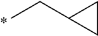 | N |
| I-106 | | 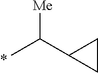 | N |
| I-107 | | 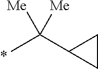 | N |
| I-108 | | 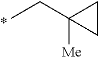 | N |
| I-109 | | 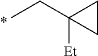 | N |
| I-110 | | 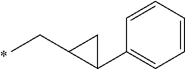 | N |
| I-111 | | 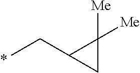 | N |
| I-112 | | 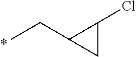 | N |
| I-113 | | 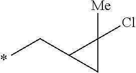 | N |
| I-114 | | 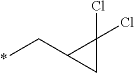 | N |
| I-115 | | 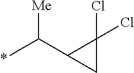 | N |
| I-116 | | 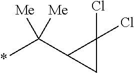 | N |
| I-117 | | 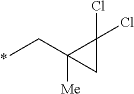 | N |
| I-118 | | 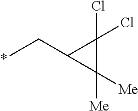 | N |
| I-119 | | 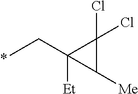 | N |
| I-120 | | 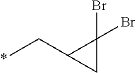 | N |

TABLE 8

| Compound No. | R1 | R2 | A |
|---|---|---|---|
| I-121 | 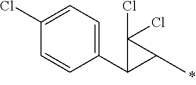 | 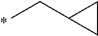 | N |
| I-122 | | 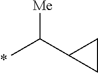 | N |
| I-123 | | 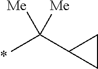 | N |
| I-124 | | 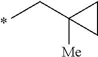 | N |
| I-125 | | 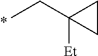 | N |
| I-126 | | 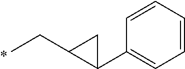 | N |
| I-127 | | 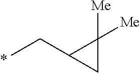 | N |
| I-128 | | 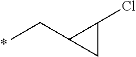 | N |
| I-129 | | 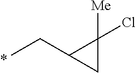 | N |
| I-130 | | 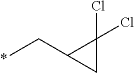 | N |
| I-131 | | 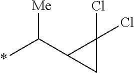 | N |
| I-132 | | 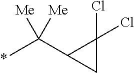 | N |
| I-133 | | 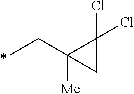 | N |
| I-134 | | 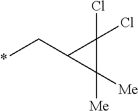 | N |
| I-135 | | 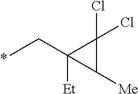 | N |
| I-136 | | 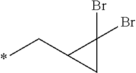 | N |

TABLE 9

| Compound No. | R1 | R2 | A |
|---|---|---|---|
| I-137 | 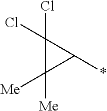 | 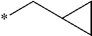 | CH |
| I-138 | | 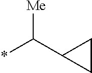 | CH |
| I-139 | | 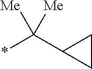 | CH |
| I-140 | | 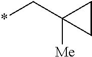 | |
| I-141 | | 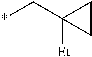 | |
| I-142 | | 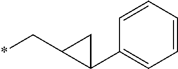 | CH |
| I-143 | | 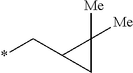 | CH |
| I-144 | | 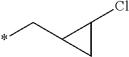 | CH |
| I-145 | | 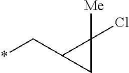 | CH |
| I-146 | | 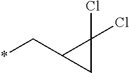 | CH |
| I-147 | | 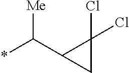 | CH |
| I-148 | | 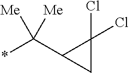 | CH |
| I-149 | | 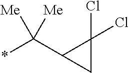 | CH |
| I-150 | | 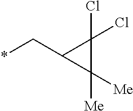 | CH |
| I-151 | | 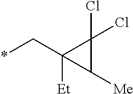 | CH |
| I-152 | | 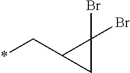 | CH |

TABLE 10

| Compound No. | R1 | R2 | A |
|---|---|---|---|
| I-153 | 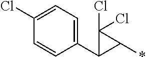 | 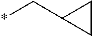 | CH |
| I-154 | | 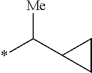 | CH |
| I-155 | | 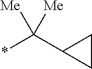 | CH |
| I-156 | | 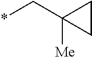 | CH |
| I-157 | | 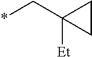 | CH |
| I-158 | | 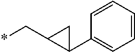 | CH |
| I-159 | | 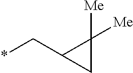 | CH |
| I-160 | | 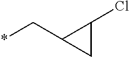 | |
| I-161 | | 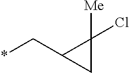 | CH |
| I-162 | | 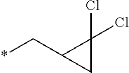 | CH |
| I-163 | | 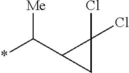 | CH |
| I-164 | | 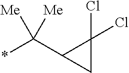 | CH |
| I-165 | | 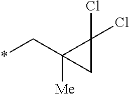 | CH |
| I-166 | | 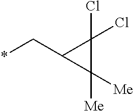 | |
| I-167 | | 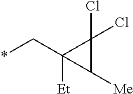 | CH |
| I-168 | | 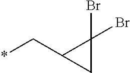 | CH |

TABLE 11

| Compound No. | R1 | R2 | A |
|---|---|---|---|
| I-169 | 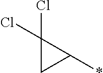 | 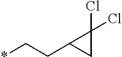 | N |
| I-170 | | 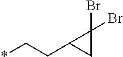 | N |
| I-171 | 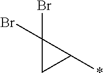 | 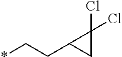 | N |
| I-172 | | 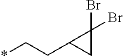 | N |
| I-173 | 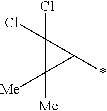 | 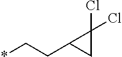 | N |
| I-174 | | 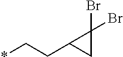 | N |
| I-175 | 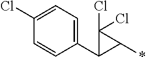 | 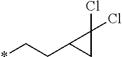 | N |
| I-176 | | 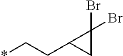 | N |
| I-177 | 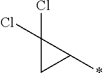 | 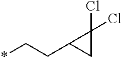 | CH |
| I-178 | | 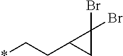 | CH |
| I-179 | 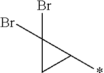 | 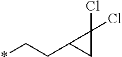 | CH |
| I-180 | | 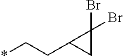 | CH |
| I-181 | 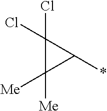 | 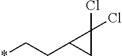 | CH |
| I-182 | | 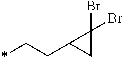 | CH |
| I-183 | 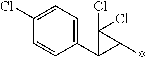 | 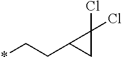 | CH |
| I-184 | | 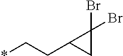 | CH |

TABLE 12

| Compound No. | R1 | R2 | A |
|---|---|---|---|
| I-185 | 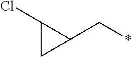 | 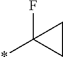 | N |
| I-186 | | 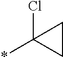 | N |
| I-187 | | 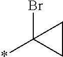 | N |
| I-188 | 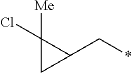 | 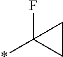 | N |
| I-189 | | 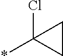 | N |
| I-190 | | 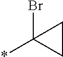 | N |
| I-191 | 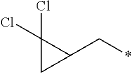 | 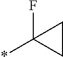 | N |
| I-192 | | 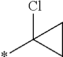 | N |
| I-193 | | 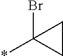 | N |
| I-194 | 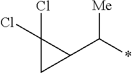 | 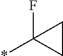 | N |
| I-195 | | 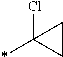 | N |
| I-196 | | 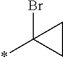 | N |

TABLE 13

| Compound No | R1 | R2 | A |
|---|---|---|---|
| I-197 | 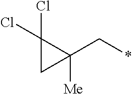 | 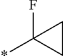 | N |
| I-198 | | 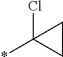 | N |
| I-199 | | 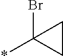 | N |
| I-200 | 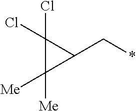 | 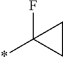 | N |
| I-201 | | 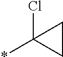 | N |
| I-202 | | 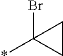 | |
| I-203 | 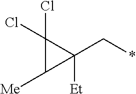 | 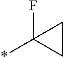 | N |
| I-204 | | 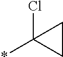 | N |
| I-205 | | 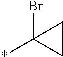 | N |
| I-206 | 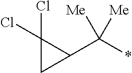 | 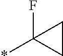 | N |
| I-207 | | 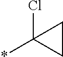 | N |
| I-208 | | 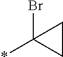 | N |
| I-209 | 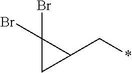 | 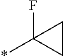 | N |
| I-210 | | 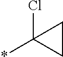 | N |
| I-211 | | 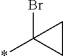 | N |

TABLE 14

| Compound No. | R1 | R2 | A |
|---|---|---|---|
| I-212 | 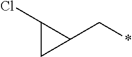 | 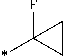 | CH |
| I-213 | | 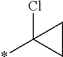 | CH |
| I-214 | | 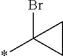 | CH |
| I-215 | 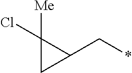 | 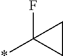 | CH |
| I-216 | | 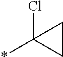 | CH |
| I-217 | | 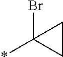 | CH |
| I-218 | 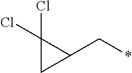 | 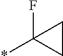 | CH |
| I-219 | | 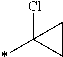 | CH |
| I-220 | | 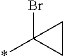 | CH |
| I-221 | 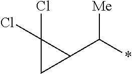 | 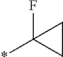 | CH |
| I-222 | | 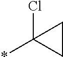 | CH |
| I-223 | | 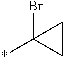 | CH |

TABLE 15

| Compound No. | R1 | R2 | A |
|---|---|---|---|
| I-224 | 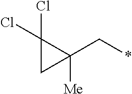 | 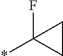 | CH |
| I-225 | | 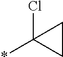 | CH |
| I-226 | | 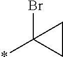 | CH |
| I-227 | 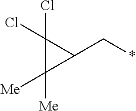 | 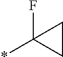 | CH |
| I-228 | | 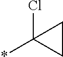 | CH |
| I-229 | | 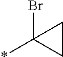 | CH |
| I-230 | 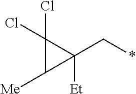 | 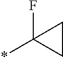 | CH |
| I-231 | | 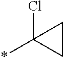 | CH |
| I-232 | | 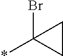 | CH |
| I-233 | 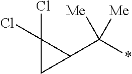 | 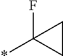 | CH |
| I-234 | | 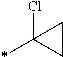 | CH |
| I-235 | | 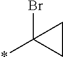 | CH |
| I-236 | 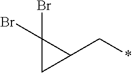 | 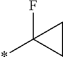 | CH |
| I-237 | | 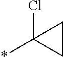 | CH |
| I-238 | | 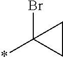 | CH |

TABLE 16

| Compound No. | R1 | R2 | A |
|---|---|---|---|
| I-239 | 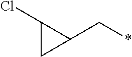 | 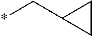 | N |
| I-240 | | 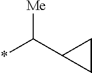 | N |
| I-241 | | 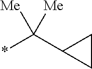 | N |
| I-242 | | 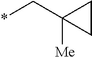 | N |
| I-243 | | 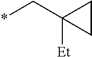 | N |
| I-244 | | 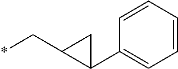 | N |
| I-245 | | 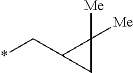 | N |
| I-246 | | 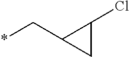 | N |
| I-247 | | 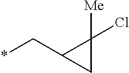 | N |
| I-248 | | 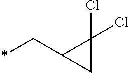 | N |
| I-249 | | 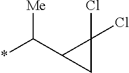 | N |
| I-250 | | 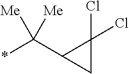 | N |
| I-251 | | 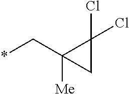 | N |
| I-252 | | 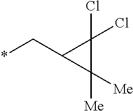 | N |
| I-253 | | 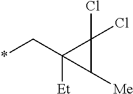 | N |
| I-254 | | 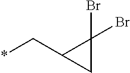 | N |

TABLE 17

| Compound No. | R1 | R2 | A |
|---|---|---|---|
| I-255 | 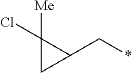 | 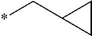 | N |
| I-256 | | 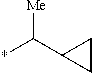 | N |
| I-257 | | 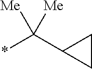 | N |
| I-258 | | 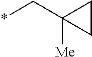 | N |
| I-259 | | 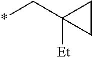 | N |
| I-260 | | 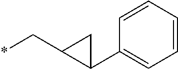 | N |
| I-261 | | 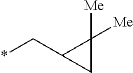 | N |
| I-262 | | 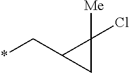 | N |
| I-263 | | 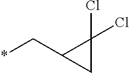 | N |
| I-264 | | 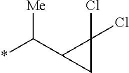 | N |
| I-265 | | 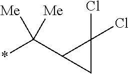 | N |
| I-266 | | 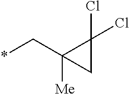 | N |
| I-267 | | 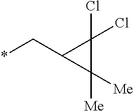 | N |
| I-268 | | 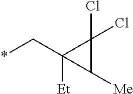 | N |
| I-269 | | 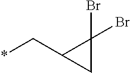 | N |

TABLE 18

| Compound No. | R1 | R2 | A |
|---|---|---|---|
| I-270 | 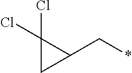 | 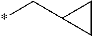 | N |
| I-271 | | 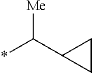 | N |
| I-272 | | 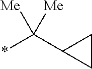 | N |
| I-273 | | 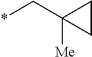 | N |
| I-274 | | 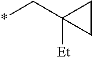 | N |
| I-275 | | 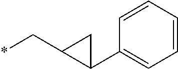 | N |
| I-276 | | 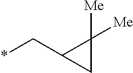 | N |
| I-277 | | 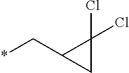 | N |
| I-278 | | 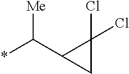 | N |
| I-279 | | 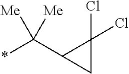 | N |
| I-280 | | 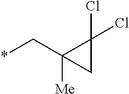 | N |
| I-281 | | 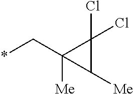 | N |
| I-282 | | 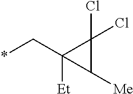 | N |
| I-283 | | 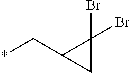 | N |

TABLE 19

| Compound No. | R1 | R2 | A |
|---|---|---|---|
| I-284 | 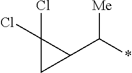 | 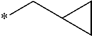 | N |
| I-285 | | 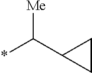 | N |
| I-286 | | 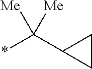 | N |
| I-287 | | 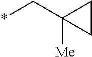 | N |
| I-288 | | 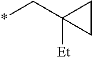 | N |
| I-289 | | 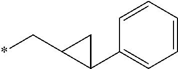 | N |
| I-290 | | 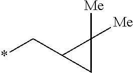 | N |
| I-291 | | 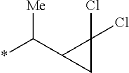 | N |
| I-292 | | 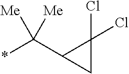 | N |
| I-293 | | 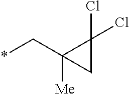 | N |
| I-294 | | 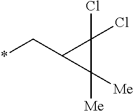 | N |
| I-295 | | 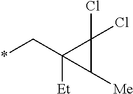 | N |
| I-296 | | 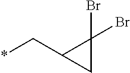 | N |

TABLE 20

| Compound No. | R1 | R2 | A |
|---|---|---|---|
| I-297 | 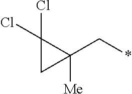 | 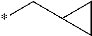 | N |
| I-298 | | 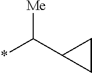 | N |
| I-299 | | 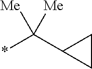 | N |
| I-300 | | 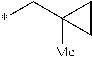 | N |
| I-301 | | 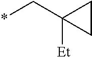 | N |
| I-302 | | 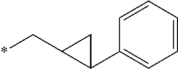 | N |
| I-303 | | 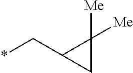 | N |
| I-304 | | 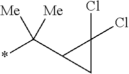 | N |
| I-305 | | 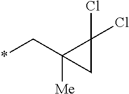 | N |
| I-306 | | 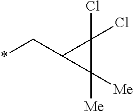 | N |
| I-307 | | 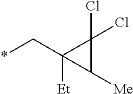 | N |
| I-308 | | 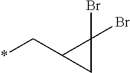 | N |

TABLE 21

| Compound No. | R1 | R2 | A |
|---|---|---|---|
| I-309 | 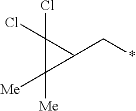 | 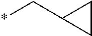 | N |
| I-310 | | 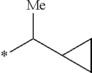 | N |
| I-311 | | 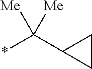 | N |
| I-312 | | 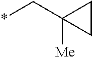 | N |
| I-313 | | 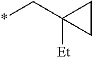 | N |
| I-314 | | 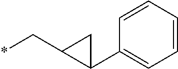 | N |
| I-315 | | 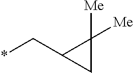 | N |
| I-316 | | 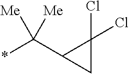 | N |
| I-317 | | 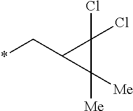 | N |
| I-318 | | 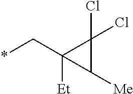 | N |
| I-319 | | 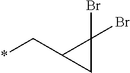 | N |

TABLE 22

| Compound No. | R1 | R2 | A |
|---|---|---|---|
| I-320 | 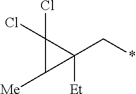 | 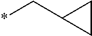 | N |
| I-321 | | 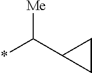 | N |
| I-322 | | 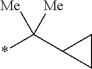 | N |
| I-323 | | 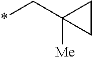 | N |
| I-324 | | 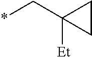 | N |
| I-325 | | 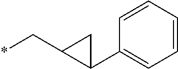 | N |
| I-326 | | 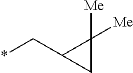 | N |
| I-327 | | 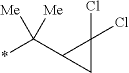 | N |
| I-328 | | 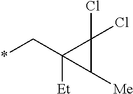 | N |
| I-329 | | 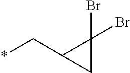 | N |

TABLE 23

| Compound No. | R1 | R2 | A |
|---|---|---|---|
| I-330 | 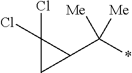 | 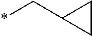 | N |
| I-331 | | 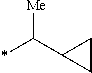 | N |
| I-332 | | 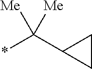 | N |
| I-333 | | 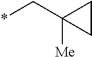 | N |
| I-334 | | 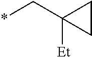 | N |
| I-335 | | 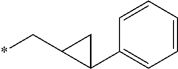 | N |
| I-336 | | 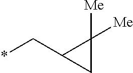 | N |
| I-337 | | 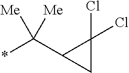 | N |
| I-338 | | 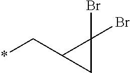 | N |
| I-339 | 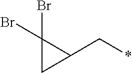 | 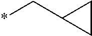 | N |
| I-340 | | 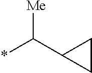 | N |
| I-341 | | 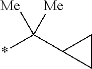 | N |
| I-342 | | 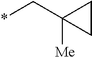 | N |
| I-343 | | 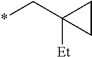 | N |
| I-344 | | 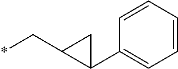 | N |
| I-345 | | 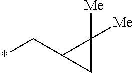 | N |
| I-346 | | 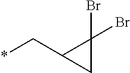 | N |

TABLE 24

| Compound | | | |
|---|---|---|---|
| No. | R1 | R2 | A |
| I-347 | 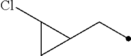 | 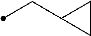 | CH |
| I-348 | | 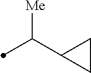 | CH |
| I-349 | | 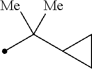 | CH |
| I-350 | | 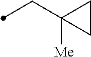 | CH |
| I-351 | | 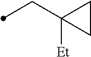 | CH |
| I-352 | | 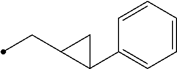 | CH |
| I-353 | | 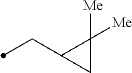 | CH |
| I-354 | | 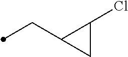 | CH |
| I-355 | | 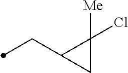 | CH |
| I-356 | | 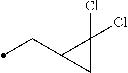 | CH |
| I-357 | | 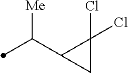 | CH |
| I-358 | | 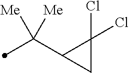 | CH |
| I-359 | | 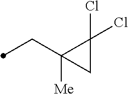 | CH |
| I-360 | | 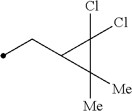 | CH |
| I-361 | | 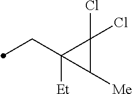 | CH |
| I-362 | | 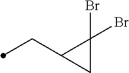 | CH |

TABLE 25

| Compound | | | |
|---|---|---|---|
| No. | R1 | R2 | A |
| I-363 | 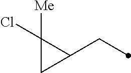 | 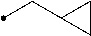 | CH |
| I-364 | | 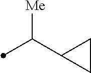 | CH |
| I-365 | | 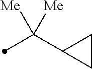 | CH |
| I-366 | | 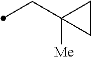 | CH |
| I-367 | | 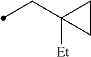 | CH |
| I-368 | | 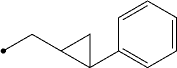 | CH |
| I-369 | | 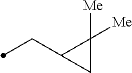 | CH |
| I-370 | | 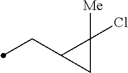 | CH |
| I-371 | | 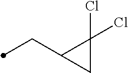 | CH |
| I-372 | | 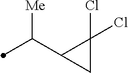 | CH |
| I-373 | | 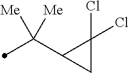 | CH |
| I-374 | | 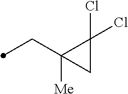 | CH |
| I-375 | | 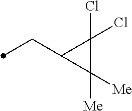 | CH |
| I-376 | | 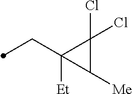 | CH |
| I-377 | | 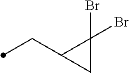 | CH |

TABLE 26

| Compound | | | |
|---|---|---|---|
| No. | R1 | R2 | A |
| I-378 | 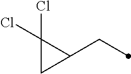 | 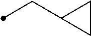 | CH |
| I-379 | | 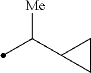 | CH |
| I-380 | | 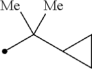 | CH |
| I-381 | | 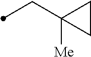 | CH |
| I-382 | | 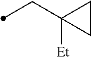 | CH |
| I-383 | | 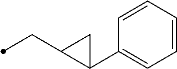 | CH |
| I-384 | | 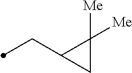 | CH |
| I-385 | | 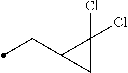 | CH |
| I-386 | | 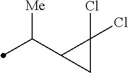 | CH |
| I-387 | | 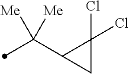 | CH |
| I-388 | | 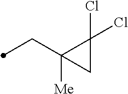 | CH |
| I-389 | | 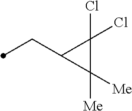 | CH |
| I-390 | | 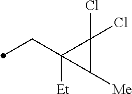 | CH |
| I-391 | | 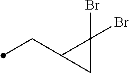 | CH |

TABLE 27

| Compound | | | |
|---|---|---|---|
| No. | R1 | R2 | A |
| I-392 | 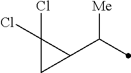 | 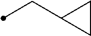 | CH |
| I-393 | | 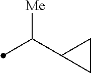 | CH |
| I-394 | | 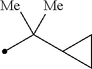 | CH |
| I-395 | | 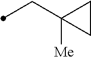 | CH |
| I-396 | | 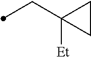 | CH |
| I-397 | | 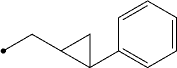 | CH |
| I-398 | | 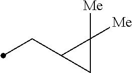 | CH |
| I-399 | | 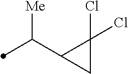 | CH |
| I-400 | | 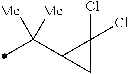 | CH |
| I-401 | | 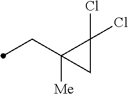 | CH |
| I-402 | | 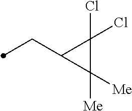 | CH |
| I-403 | | 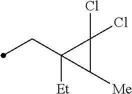 | CH |
| I-404 | | 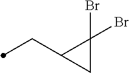 | CH |

TABLE 28

| Compound | | | |
|---|---|---|---|
| No. | R1 | R2 | A |
| I-405 | 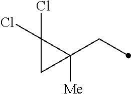 | 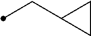 | CH |
| I-406 | | 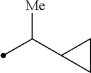 | CH |
| I-407 | | 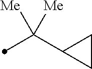 | CH |
| I-408 | | 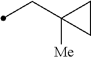 | CH |
| I-409 | | 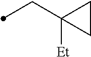 | CH |
| I-410 | | 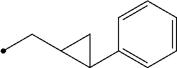 | CH |
| I-411 | | 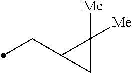 | CH |
| I-412 | | 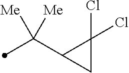 | CH |
| I-413 | | 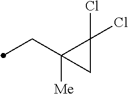 | CH |
| I-414 | | 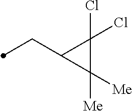 | CH |
| I-415 | | 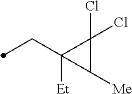 | CH |
| I-416 | | 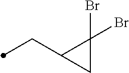 | CH |

TABLE 29

| Compound | | | |
|---|---|---|---|
| No. | R1 | R2 | A |
| I-417 | 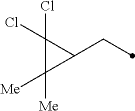 | 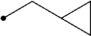 | CH |
| I-418 | | 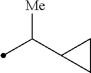 | CH |
| I-419 | | 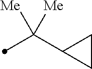 | CH |
| I-420 | | 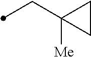 | CH |
| I-421 | | 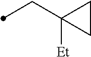 | CH |
| I-422 | | 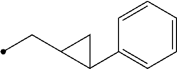 | CH |
| I-423 | | 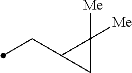 | CH |
| I-424 | | 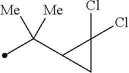 | CH |
| I-425 | | 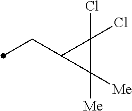 | CH |
| I-426 | | 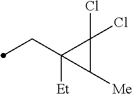 | CH |
| I-427 | | 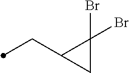 | CH |

TABLE 30

| Compound | | | |
|---|---|---|---|
| No. | R1 | R2 | A |
| I-428 | 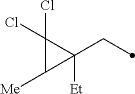 | 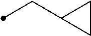 | CH |
| I-429 | | 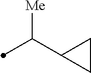 | CH |
| I-430 | | 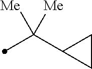 | CH |
| I-431 | | 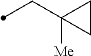 | CH |
| I-432 | | 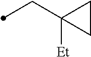 | CH |
| I-433 | | 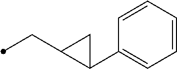 | CH |
| I-434 | | 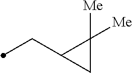 | CH |
| I-435 | | 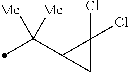 | CH |
| I-436 | | 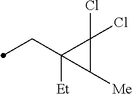 | CH |
| I-437 | | 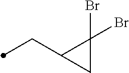 | CH |

TABLE 31

| Compound | | | |
|---|---|---|---|
| No. | R1 | R2 | A |
| I-438 | 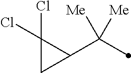 | 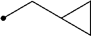 | CH |
| I-439 | | 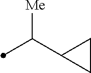 | CH |
| I-440 | | 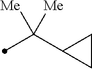 | CH |
| I-441 | | 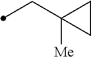 | CH |
| I-442 | | 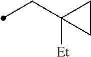 | CH |
| I-443 | | 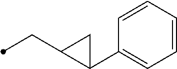 | CH |
| I-444 | | 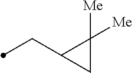 | CH |
| I-445 | | 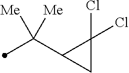 | CH |
| I-446 | | 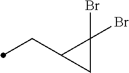 | CH |
| I-447 | 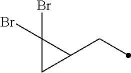 | 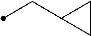 | CH |
| I-448 | | 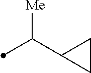 | CH |
| I-449 | | 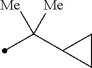 | CH |
| I-450 | | 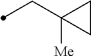 | CH |
| I-451 | | 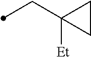 | CH |
| I-452 | | 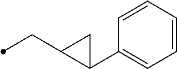 | CH |
| I-453 | | 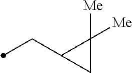 | CH |
| I-454 | | 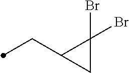 | CH |

TABLE 32

| Compound | | | |
|---|---|---|---|
| No. | R1 | R2 | A |
| I-455 | 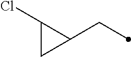 | 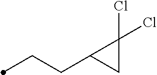 | N |
| I-456 | | 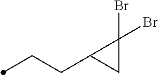 | N |
| I-457 | 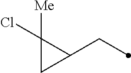 | 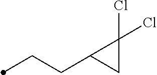 | N |
| I-458 | | 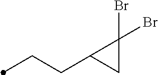 | N |
| I-459 | 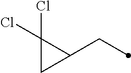 | 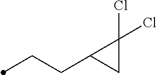 | N |
| I-460 | | 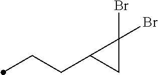 | N |
| I-461 | 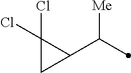 | 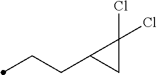 | N |
| I-462 | | 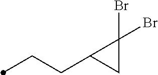 | N |
| I-463 | 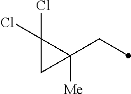 | 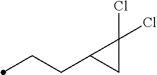 | N |
| I-464 | | 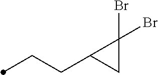 | N |
| I-465 | 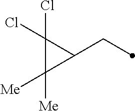 | 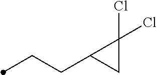 | N |
| I-466 | | 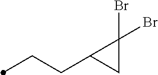 | N |
| I-467 | 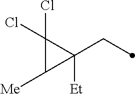 | 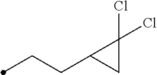 | N |
| I-468 | | 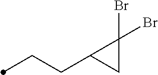 | N |
| I-469 | 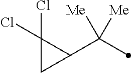 | 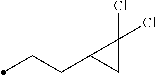 | N |
| I-470 | | 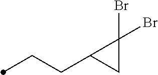 | N |
| I-471 | 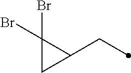 | 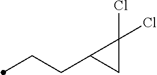 | N |
| I-472 | | 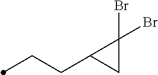 | N |

TABLE 33

| Compound | | | |
|---|---|---|---|
| No. | R1 | R2 | A |
| I-473 | 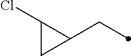 | 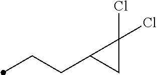 | CH |
| I-474 | | 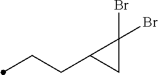 | CH |
| I-475 | 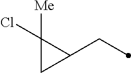 | 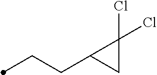 | CH |
| I-476 | | 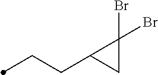 | CH |
| I-477 | 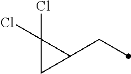 | 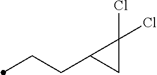 | CH |
| I-478 | | 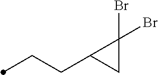 | CH |
| I-479 | 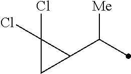 | 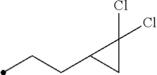 | CH |
| I-480 | | 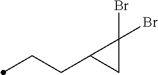 | CH |
| I-481 | 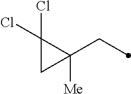 | 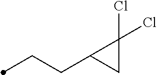 | CH |
| I-482 | | 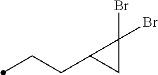 | CH |
| I-483 | 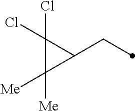 | 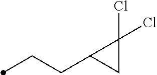 | CH |
| I-484 | | 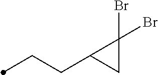 | CH |
| I-485 | 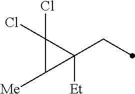 | 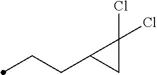 | CH |
| I-486 | | 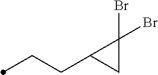 | CH |
| I-487 | 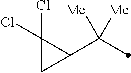 | 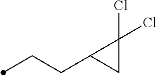 | CH |
| I-488 | | 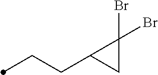 | CH |
| I-489 | 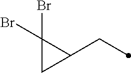 | 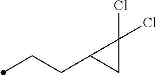 | CH |
| I-490 | | 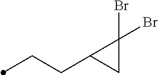 | CH |

TABLE 34

| Compound | | | |
|---|---|---|---|
| No. | R1 | R2 | A |
| I-600 | 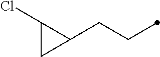 | 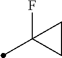 | N |
| I-601 | | 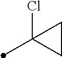 | N |
| I-602 | | 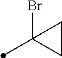 | N |
| I-603 | 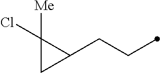 | 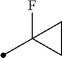 | N |
| I-604 | | 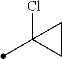 | N |
| I-605 | | 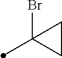 | N |
| I-606 | 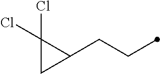 | 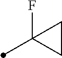 | N |
| I-607 | | 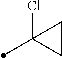 | N |
| I-608 | | 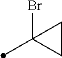 | N |
| I-609 | 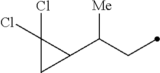 | 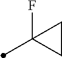 | N |
| I-610 | | 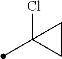 | N |
| I-611 | | 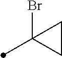 | N |

TABLE 35

| Compound | | | |
|---|---|---|---|
| No. | R1 | R2 | A |
| I-612 | 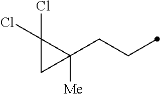 | 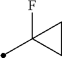 | N |
| I-613 | | 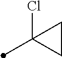 | N |
| I-614 | | 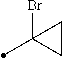 | N |
| I-615 | 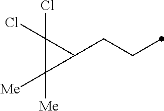 | 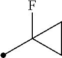 | N |
| I-616 | | 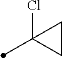 | N |
| I-617 | | 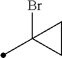 | N |
| I-618 | 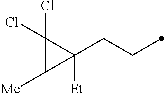 | 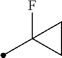 | N |
| I-619 | | 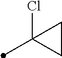 | N |
| I-620 | | 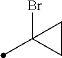 | N |
| I-621 | 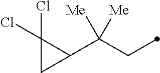 | 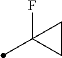 | N |
| I-622 | | 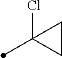 | N |
| I-623 | | 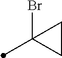 | N |
| I-624 | 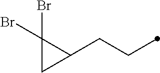 | 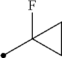 | N |
| I-625 | | 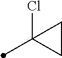 | N |
| I-626 | | 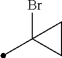 | N |

TABLE 36

| Compound | | | |
|---|---|---|---|
| No. | R1 | R2 | A |
| I-630 | 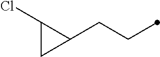 | 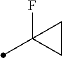 | CH |
| I-631 | | 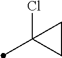 | CH |
| I-632 | | 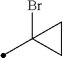 | CH |
| I-633 | 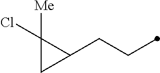 | 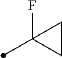 | CH |
| I-634 | | 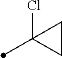 | CH |
| I-635 | | 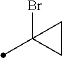 | CH |
| I-636 | 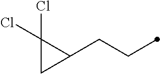 | 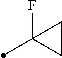 | CH |
| I-637 | | 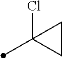 | CH |
| I-638 | | 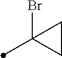 | CH |
| I-639 | 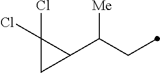 | 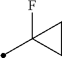 | CH |
| I-640 | | 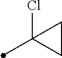 | CH |
| I-641 | | 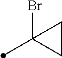 | CH |

TABLE 37

| Compound | | | |
|---|---|---|---|
| No. | R1 | R2 | A |
| I-642 | 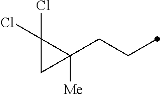 | 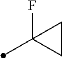 | CH |
| I-643 | | 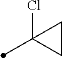 | CH |
| I-644 | | 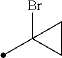 | CH |
| I-645 | 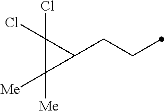 | 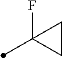 | CH |
| I-646 | | 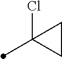 | CH |
| I-647 | | 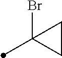 | CH |
| I-648 | 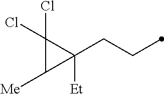 | 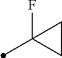 | CH |
| I-649 | | 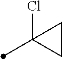 | CH |
| I-650 | | 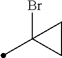 | CH |
| I-651 | 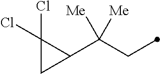 | 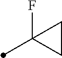 | CH |
| I-652 | | 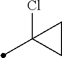 | CH |
| I-653 | | 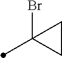 | CH |
| I-654 | 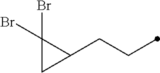 | 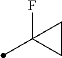 | CH |
| I-655 | | 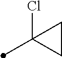 | CH |
| I-656 | | 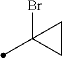 | CH |

In the tables, each of R¹ and R² is indicated with a dot for the binding position thereof. That is, it should be understood that between the carbon atom to which the dot is attached and the carbon atom to which a hydroxyl group is bound in Compound (I) a carbon-carbon bond is formed.

Among the typical examples described above, a compound having a cyclopropyl group or a (cyclopropyl) C1-C4 alkyl group in which one to two halogen atoms are substituted on either one of R¹ and R² is more preferred.

A compound having a cyclopropyl group or a (cyclopropyl) C1-C4 alkyl group in which one to two halogen atoms are substituted on both of R¹ and R² is further preferred.

It is especially preferred that one of R¹ and R² is a cyclopropyl group substituted with one halogen atom and the other is a (cyclopropyl) C1-C4 alkyl group substituted with two halogen atoms.
Herein, the cyclopropyl group substituted with one halogen atom which is preferred may be, for example, 1-fluorocyclopropyl, a 1-chlorocyclopropyl group, and a 1-bromocyclopropyl group, and 1-fluorocyclopropyl and a 1-chlorocyclopropyl group are more preferred, with a 1-chlorocyclopropyl group being especially preferred. The (cyclopropyl) C1-C4 alkyl group substituted with two halogen atoms may be, for example, a (2,2-difluorocyclopropyl)methyl group, a 2-(2,2-difluorocyclopropyl)ethyl group, a 3-(2,2-difluorocyclopropyl)propyl group, a (2,2-dichlorocyclopropyl)methyl group, a 2-(2,2-dichlorocyclopropyl)ethyl group, a 3-(2,2-dichlorocyclopropyl)propyl group, a 4-(2,2-dichlorocyclopropyl)butyl group, a (2,2-dibromocyclopropyl)methyl group, a 2-(2,2-dibromocyclopropyl)ethyl group, a 3-(2,2-dibromocyclopropyl)propyl group, a 4-(2,2-dibromocyclopropyl)butyl group, a (2,2-diiodocyclopropyl)methyl group,
those more preferred being (2,2-dihalocyclopropyl) C1-C2 alkyl groups such as a (2,2-difluorocyclopropyl)methyl group, a 2-(2,2-difluorocyclopropyl)ethyl group, a (2,2-dichlorocyclopropyl)methyl group, a 2-(2,2-dichlorocyclopropyl)ethyl group, a (2,2-dibromocyclopropyl)methyl group, and a 2-(2,2-dibromocyclopropyl)ethyl group, and those especially preferred may be, for example, a (2,2-dichlorocyclopropyl)methyl group, a 2-(2,2-dichlorocyclopropyl)ethyl group, a (2,2-dibromocyclopropyl)methyl group, and a 2-(2,2-dibromocyclopropyl)ethyl group.

2. Methods for Producing Azole Derivatives

The method for producing Compound (I) is described below. Solvents, bases, acids, and the like employed in each step in the production method according to the invention may be those listed below unless otherwise specified.

(1) Solvents

While the solvent employed is not limited particularly, those which may be exemplified include halogenated hydrocarbons such as dichloromethane, chloroform, and dichloroethane, aromatic hydrocarbons such as benzene, toluene, and xylene, aliphatic hydrocarbons such as petroleum ether, hexane, and methylcyclohexane, amides such as N,N-dimethylformamide, N,N-dimethylacetamide, and N-methyl-2-pyrrolidinone, ethers such as diethyl ether, tetrahydrofuran, and dioxane, alcohols such as methanol and ethanol. Otherwise, solvents may be, for example, water, carbon disulfide, acetonitrile, ethyl acetate, pyridine, and dimethyl sulfoxide. Two or more of these solvents may be employed in combination.

One which may also be exemplified as a solvent is a solvent composition consisting of solvents which do not form a homogenous layer with each other. For example, to a reaction mixture, quaternary ammonium salts such as tetrabutylammonium salt, trimethylbenzylammonium salt, and triethylbenzylammonium salt, and a phase transfer catalyst such as a crown ether and its analogues are added to effect the reaction thereof. In such a case, the solvents employed are not limited, while the oily phase may consists of benzene, chloroform, dichloromethane, hexane, toluene, tetrahydrofuran and the like.

(2) Bases and Acids

To the solvent described above, a base or an acid may be added.

While the base employed is not limited particularly, it may be, for example, a carbonate of an alkaline metal such as sodium carbonate, sodium hydrogen carbonate, potassium carbonate, and potassium hydrogen carbonate; a carbonate of an alkaline earth metal such as calcium carbonate and barium carbonate; a hydroxide of an alkaline metal such as sodium hydroxide and potassium hydroxide; an alkaline metal such as lithium, sodium, and potassium; an alkoxide of an alkaline metal such as sodium methoxide, sodium ethoxide, and potassium t-butoxide; an alkaline metal hydride such as sodium hydride, potassium hydride, and lithium hydride; an organometallic compound of an alkaline metal such as n-butyl lithium and methyl magnesium bromide; an alkaline metal such as sodium, potassium, and lithium; an alkaline metal amide such as lithium diisopropyl amide; and an organic amine such as triethylamine, pyridine, 4-dimethylaminopyridine, N,N-dimethylaniline, and 1,8-diazabicyclo-7-[5.4.0] undecene.

While the acid employed is not limited particularly either, it may be, for example, an inorganic acid such as hydrochloric acid, hydrobromic acid, hydroiodic acid, and sulfuric acid, an organic acid such as formic acid, acetic acid, butyric acid, and p-toluenesulfonic acid, a Lewis acid such as lithium chloride, lithium bromide, rhodium chloride, zinc chloride, iron chloride, and aluminum chloride.

(3) First Method for Producing Compound (I)

(3-1) Step A1

One embodiment of this production method comprises a step for reacting an oxirane compound represented by Formula (II) shown below with a 1,2,4-triazole or imidazole compound represented by Formula (III) shown below (Step A1) (see Scheme (1) shown below). Hereinafter, the oxirane compound represented by Formula (II) is referred to as “Compound (II)”, while the 1,2,4-triazole or imidazole compound represented by Formula (III) is referred to as “Compound (III)”.

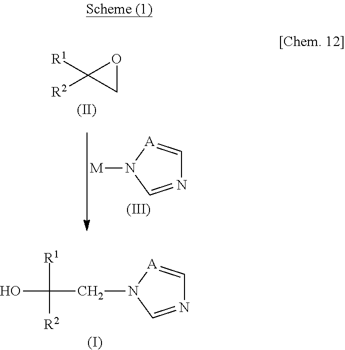

Herein, the contexts of the definitions of R¹, R², and A are as defined above.

M denotes a hydrogen atom or an alkaline metal.

In this step, a carbon atom in the oxirane ring in Compound (II) is reacted with Compound (III) to form a carbon-nitrogen bond between the carbon atom in the oxirane ring in Compound (II) and a nitrogen atom in Compound (III).

The solvent employed here is not limited particularly, and may be, for example, amides such as N-methylpyrrolidone and N,N-dimethylformamide.

The amount of Compound (III) employed per mole of Compound (II) is usually 0.5 to 10 moles, preferably 0.8 to 5 moles. A base may be added if desired. In such a case, the amount of the base employed per mole of Compound (III) is usually 0 to 10 moles, preferably 0.5 to 5 moles.

The reaction temperature and the reaction time may appropriately be selected depending on the types of the solvent, the base and the like which are employed. The reaction temperature is preferably 0 degrees C. to 250 degrees C., more preferably 10 degrees C. to 150 degrees C. The reaction time is preferably 0.1 hour to several days, more preferably 0.5 hour to 2 days.

(3-2) Step A2

As a preferred first synthetic method of Compound (II) employed in Step A1, a method for reacting a halohydrin compound (hereinafter referred to as “Compound (VI)”) represented by Formula (VI) in a solvent in the presence of a base may be exemplified (see Scheme (2) shown below).

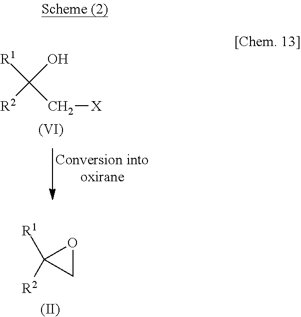

Herein, the contexts of the definitions of R¹ and R² are as defined above. X denotes a halogen atom.

The base employed preferably includes, but is not limited to, a hydroxide of an alkaline metal or an alkaline earth metal such as sodium hydroxide, potassium hydroxide, and calcium hydroxide; a carbonate or a hydrogen carbonate of an alkaline metal such as sodium carbonate and potassium carbonate.

The amount of the base is 0.5 to 20 moles, preferably 0.8 to 5 moles per mole of Compound (VI).

The solvent includes, but not limited to, alcohols such as methanol, ethanol, and isopropanol; ethers such as diethyl ether, tetrahydrofuran, and dioxane; amides such as N,N-dimethylformamide, N,N-dimethylacetamide, and N-methyl-2-pyrrolidinone; hydrocarbons such as n-hexane, methylcyclohexane, benzene, toluene, and xylene; halogenated hydrocarbons such as dichloroethane and chloroform; as well as a solvent mixture thereof. When using an aqueous solution of a base together with a hydrophobic solvent, a phase transfer catalyst such as quaternary ammonium salts, crown ether and its analogues may be added to the reaction mixture thereby effecting the reaction. The quaternary ammonium salts may be, for example, tetrabutylammonium salt, trimethylbenzylammonium salt and triethylbenzylammonium salt.

(3-3) Step A3

Compound (VI) used in Step A2 can be produced by subjecting the carbonyl group of a compound represented by Formula (VII) (hereinafter referred to as “Compound (VII)”) to nucleophilic addition of a compound represented by Formula (IV) (hereinafter referred to as “Compound (IV)”) thereby forming a carbon-carbon bond (see Scheme (3) shown below).

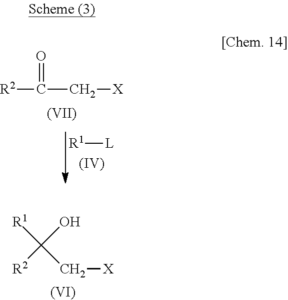

Herein, the contexts of the definitions of R¹, R², and X are as defined above.

L may be, for example, an alkaline metal, an alkaline earth metal-Q₁ (Qᵢ is an halogen atom), a ½ (Cu alkaline metal), and a zinc-Q₂ (Q₂ is a halogen atom), any of which can be employed. The alkaline metal may be, for example, lithium, sodium, and potassium, with lithium being preferred. The alkaline earth metal may be, for example, magnesium.

While the solvent employed is not limited particularly as long as it is a solvent which is inert under the condition of the reaction, it may be, for example, ethers such as diethyl ether, tetrahydrofuran, and dioxane, aromatic hydrocarbons such as benzene, toluene, and xylene. When using an aqueous solution together with a hydrophobic solvent, quaternary ammonium salts such as tetrabutylammonium salt, trimethylbenzylammonium salt, and triethylbenzylammonium salt, and a phase transfer catalyst such as a crown ether and its analogues may be added to the reaction mixture thereby effecting the reaction.

The amount of Compound (IV) employed per mole of Compound (VII) is usually 0.5 to 10 moles, preferably 0.8 to 5 moles. Compound (IV) prepared immediately before use is preferred. There may be a case that the reaction can be conducted while allowing Compound (IV) to be produced in the reaction system, which is preferred especially when L is a zinc-Q₂ (Q₂ is a halogen atom).

It is also possible to add a Lewis acid if desired. The amount of the Lewis acid employed per mole of Compound (IV) is usually more than 0 and not more than 5 moles, preferably 0.1 to 2 moles. The Lewis acid employed may be, for example, aluminum chloride, zinc chloride, and cerium chloride.

The reaction temperature and the reaction time may appropriately be selected depending on the types of the solvent, Compound (VII) and Compound (IV) and the like which are employed. The reaction temperature is preferably −80 degrees C. to 200 degrees C., more preferably −50 degrees C. to 100 degrees C. The reaction time is preferably 0.1 to 12 hours, more preferably 0.5 to 6 hours.

Compound (IV) and Compound (VII) employed here may be commercially available compounds or those which can be produced by existing technologies.

(3-4) Step A2a

Among Compounds (II) employed in Step A1, a compound having a gem-dihalocyclopropane structure in its molecule represented by Formula (II-a) (hereinafter referred to as “Compound (II-a)”) can be obtained by a preferred second synthetic method shown below. That is, the synthesis can be conducted starting from an oxirane compound having a double bond in its molecule represented by Formula (VIII) (hereinafter referred to as “Compound (VIII)”) by a reaction of a trihalomethane and a base such as sodium hydroxide. Alternatively, the synthesis can be conducted starting from Compound (VIII) by an addition reaction of a halocarbene produced for example by a thermal decomposition of a trihaloacetate. These reactions are indicated in Scheme (4) shown below.

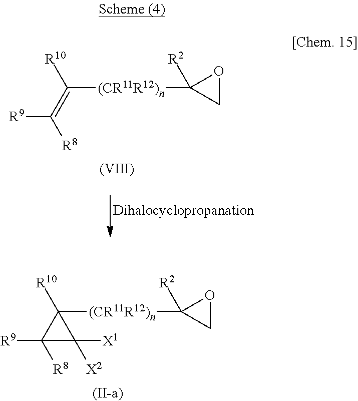

Herein, the context of the definition of R² is as defined above.

R⁸, R⁹, R¹⁰, R¹¹, and R¹² each independently denotes a hydrogen atom, a halogen atom, a C1-C4 alkyl group, a C1-C4 haloalkyl group, a C3-C6 cycloalkyl group, an aryl group, or an arylalkyl group (alkyl moiety carbon chain being C1-C3). In the cases of the aryl group and the arylalkyl group, the phenyl moiety may be substituted with a halogen atom, a C1-C4 alkyl group, a C1-C4 haloalkyl group, a C1-C4 alkoxy group, or a C1-C4 haloalkoxy group.

n denotes an integer of 0 to 4. Although a plurality of R¹¹ and R¹² will exist here when n is 2 or more, their contexts of the definitions each independently indicates the contexts of the definitions of R¹¹ and R¹². X¹ and X² each independently denotes a halogen atom.

As a preferred method for synthesizing Compound (II-a), a synthetic method by a reaction of a trihalomethane and a base such as sodium hydroxide is described below.

The trihalomethane employed may be, for example, chloroform, bromoform, chlorodifluoromethane, dichlorofluoromethane, and dibromofluoromethane. While the amount of the trihalomethane per mole of Compound (VIII) is not limited particularly, it is usually 0.5 to 1000 moles, preferably 0.8 to 100 moles.

The solvent may be the trihalomethane itself or may be other solvents which are inert to the reaction such as dichloromethane and toluene.

When an aqueous solution such as an aqueous solution of sodium hydroxide is employed upon addition of a base, it is preferred to use a phase transfer catalyst. The phase transfer catalyst is not limited particularly, and may be, for example, quaternary ammonium salts such as tetramethylammonium chloride, tetrabutylammonium bromide, cetyltrimethylammonium bromide, benzyltriethyllammonium chloride, benzyltrimethylammonium chloride, tertiary amines such as triethylamine and tripropylamine. The amount of the phase transfer catalyst employed per mole of Compound (VIII) is usually 0.001 moles to 5 moles, preferably 0.01 moles to 2 moles.

Although the base employed is not limited either, an alkaline metal hydroxide such as sodium hydroxide and potassium hydroxide is employed preferably, mostly in the form of an aqueous solution. The amount of the base employed per mole of Compound (VIII) is usually 0.1 moles to 100 moles, preferably 0.8 moles to 50 moles. In such a case, the concentration of the aqueous solution of the alkaline metal hydroxide is usually 10% to saturation of the aqueous solution, preferably 30% to saturation of the aqueous solution.

The reaction temperature is usually 0 degrees C. to 200 degrees C., preferably 10 degrees C. to 150 degrees C. The reaction time is 0.1 hour to several days, preferably 0.2 hour to 2 days.

(3-5) Step A4

Compound (VIII) employed in Step A2a can be obtained by a preferred first synthetic method shown below. Compound (VII) described above is reacted first with an organometallic compound represented by Formula (X) (hereinafter referred to as “Compound X”) to effect a nucleophilic addition reaction by the organometallic compound toward a carbonyl carbon atom in Compound (VII) thereby forming a carbon-carbon bond. As a result, a halohydrin compound represented by Formula (IX) (hereinafter referred to as “Compound (IX)”) is obtained. Then, Compound (IX) is converted into an oxirane in the presence of a base to obtain Compound (VIII) (see Scheme (5) shown below).

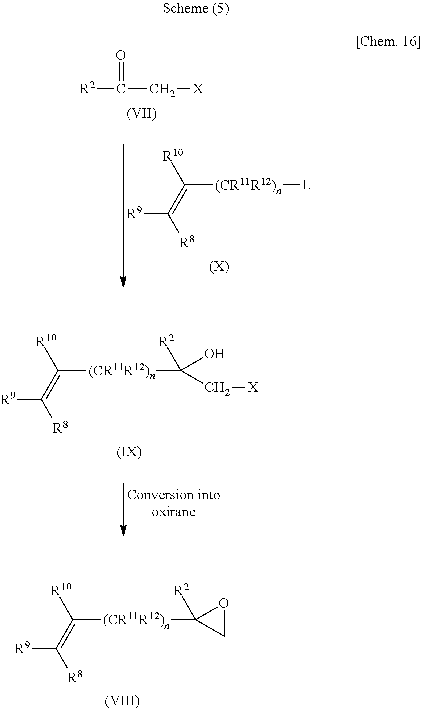

Herein, the contexts of the definitions of R², R⁸, R⁹, R¹⁰, R¹¹, R¹², L, X, and n are as defined above.

The reaction for reacting Compound (VII) with Compound (X) to obtain Compound (IX) is described below.

While the solvent employed is not limited particularly as long as it is an inert solvent, it may be, for example, ethers such as diethyl ether, tetrahydrofuran, and dioxane, aromatic hydrocarbons such as benzene, toluene, and xylene. These solvents may be used in combination. When water is used in the reaction, it may be used as being admixed with an organic solvent, and, when used together with a hydrophobic organic solvent, a phase transfer catalyst such as quaternary ammonium salts, crown ether and its analogues may be added if desired to the reaction mixture thereby effecting the reaction. The quaternary ammonium salts may be, for example, tetrabutylammonium salt, trimethylbenzylammonium salt and triethylbenzylammonium salt.

The amount of Compound (X) per mole of Compound (VII) is usually 0.5 to 10 moles, preferably 0.8 to 5 moles. Compound (X) prepared immediately before use is preferred. There may be a case where the reaction can be conducted while allowing Compound (X) to be produced in the reaction system, which is preferred especially when L is a zinc-Q₂ (Q₂ is a halogen atom).

It is also possible to add a Lewis acid if desired, and in such a case the amount of the Lewis acid employed per mole of Compound (VII) is usually more than 0 and not more than 5 moles, preferably 0.1 to 2 moles. The Lewis acid employed may be, for example, aluminum chloride, zinc chloride, and cerium chloride.

The reaction temperature and the reaction time may appropriately be selected depending on the types of the solvent, Compound (VII) and Compound (X) and the like which are employed. The reaction temperature is preferably −100 degrees C. to 200 degrees C., more preferably −70 degrees C. to 100 degrees C. The reaction time is preferably 0.1 to 12 hours, more preferably 0.5 hour to 6 hours.

The conversion of Compound (IX) to an oxirane in this step may be conducted under the condition similar to that for the synthesis from Compound (VI) to Compound (II) in Step A2.

Compound (X) employed here may be commercially available compounds or those which can be produced by an existing synthetic technology such as conversion from a halogenated alkenyl compound into an organometallic reagent. For example, as an example of a method for conducting the reaction while allowing Compound (X-a) to be produced in the reaction system in the case where L in Compound (X) is a zinc-Q₂ (Q₂ is a halogen atom), a preferred method indicated in Scheme (6) shown below can be employed.

For producing Compound (X-a), a method for production in the system from a halogenated alkenyl represented by Compound (XVII) and zinc is preferred. That is, the preparation is accomplished by mixing in the solvent in the presence of Compound (VII).

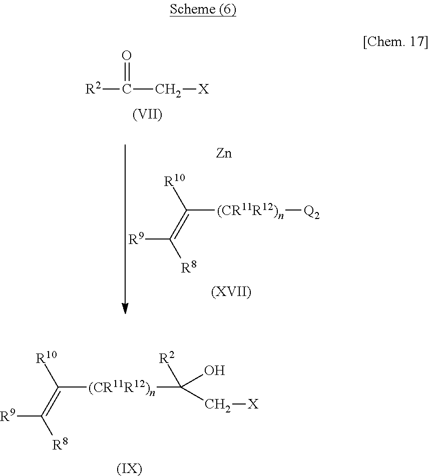

Herein, the contexts of the definitions of R², R⁸, R⁹, R¹⁰, R¹¹, R¹², Q₂, X, and n are as defined above.

While the solvent employed is not limited particularly, it may be, for example, organic solvents including ethers such as diethyl ether, tetrahydrofuran, and dioxane, aromatic hydrocarbons such as benzene, toluene, and xylene. When water is used in the reaction, it may be used as being admixed with an organic solvent, and, when used together with a hydrophobic organic solvent, a phase transfer catalyst such as quaternary ammonium salts, crown ether and its analogues may be added if desired to the reaction mixture thereby effecting the reaction. The quaternary ammonium salts may be, for example, tetrabutylammonium salt, trimethylbenzylammonium salt and triethylbenzylammonium salt.

In an example of a further preferred embodiment, under the condition allowing the contact between an organic solvent, such as a tetrahydrofuran, containing Compound (VII) and an aqueous solution containing an additive which promotes the activation of zinc such as a salt containing a halogenated hydrogen such as ammonium chloride and ammonium bromide or a halogenated hydrogen such as hydrogen chloride or hydrogen bromide, the halogenated alkenyl represented by Compound (XVII) and zinc are mixed.

In such a case, the amount of Compound (XVII) employed per mole of Compound (VII) is usually 0.5 to 20 moles, preferably 0.8 to 10 moles. The amount of zinc employed per mole of Compound (VII) is usually 0.5 to 20 moles, preferably 0.8 to 10 moles.
The reaction temperature is preferably 0 degrees C. to 150 degrees C., more preferably 5 degrees C. to 100 degrees C. The reaction time is preferably 0.1 hour to 24 hours, more preferably 0.5 hour to 12 hours.

Compound (VII) employed in this step may be those which can be produced by existing synthetic technologies.

(3-6) Step A4a

Among Compounds (VIII) employed in Step A2a, an oxirane compound represented by Formula (VIII-a) (hereinafter referred to as “Compound (VIII-a)”) can be obtained by a preferred second synthetic method shown below. That is, a methyl ketone compound represented by Formula (XV) (hereinafter referred to as “Compound (XV)”) is reacted in the presence of a base with a dialkyl carbonate compound represented by Formula (XVI) (hereinafter referred to as “Compound (XVI)”) to obtain a keto ester compound represented by Formula (XIII) (hereinafter referred to as “Compound (XIII)”). Then, in the presence of a base, a nucleophilic replacement reaction of the carbon atom to which an alkoxycarbonyl group is bound in Compound (XIII) onto a halogenated alkenyl compound represented by Formula (XIV) (hereinafter referred to as “Compound (XIV)”) is effected to produce a carbon-carbon bond, thereby obtaining an alkenylated keto ester compound represented by Formula (XII) (hereinafter referred to as “Compound (XII)”). Subsequently, Compound (XII) is hydrolyzed/decarbonated to obtain a carbonyl compound represented by Formula (XI) (hereinafter referred to as “Compound (XI)”). Finally, Compound (XI) is converted into an oxirane to obtain Compound (VIII-a). These reactions are indicated in Scheme (7) shown below.

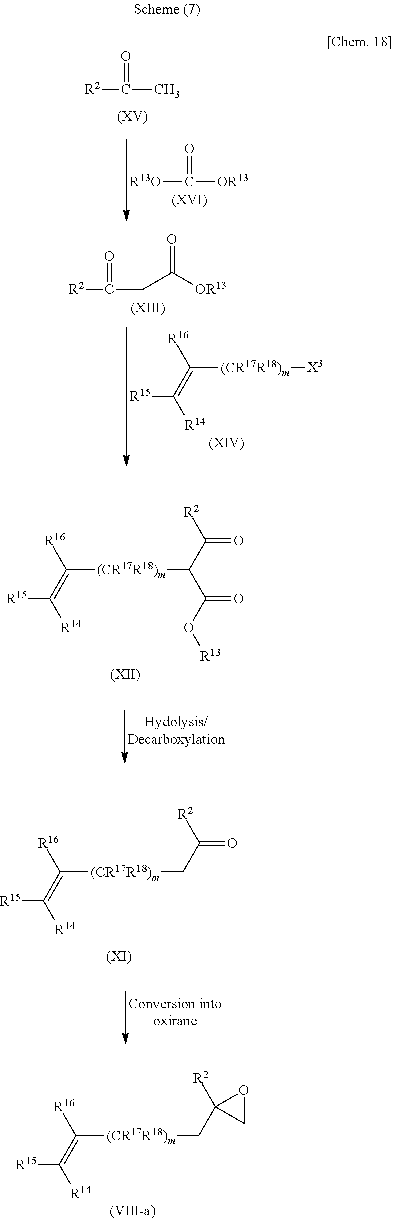

Herein, the context of the definition of R² is as defined above.

R¹³ denotes a C1-C4 alkyl group. R¹⁴, R¹⁵, R¹⁶, R¹⁷, and R¹⁸ each independently denotes a hydrogen atom, a halogen atom, a C1-C4 alkyl group, a C1-C4 haloalkyl group, a C3-C6 cycloalkyl group, an aryl group, or an arylalkyl group (alkyl moiety carbon chain being C1-C3). In the cases of the aryl group and the arylalkyl group, the phenyl moiety may be substituted further with a halogen atom, a C1-C4 alkyl group, a C1-C4 haloalkyl group, a C1-C4 alkoxy group, or a C1-C4 haloalkoxy group.

m denotes an integer of 1 to 3. Although a plurality of R¹⁷ and R¹⁸ will exist here when m is 2 or more, their contexts of the definitions each independently indicates the contexts of the definitions of R¹⁷ and R¹⁸.

X³ denotes a halogen atom.

First, a reaction for reacting Compound (XV) in the presence of a base with Compound (XVI) to obtain Compound (XIII) is described.

This reaction can be conducted in a solvent or using Compound (XVI) as a solvent.

The amount of Compound (XVI) employed per mole of Compound (XV) is usually 0.5 to 20 moles, preferably 0.8 to 10 moles.

The base employed may be, for example, but is not limited to, alkaline metal hydrides such as sodium hydride, alkaline metal alkoxides such as sodium methoxide, sodium ethoxide, and potassium t-butoxide. The amount of the base employed per mole of Compound (XV) is usually 0.5 to 10 moles, preferably 0.8 to 5 moles.

The reaction temperature is usually 0 degrees C. to 250 degrees C., preferably room temperature to 150 degrees C. The reaction time is usually 0.1 hour to several days, preferably 0.5 hour to 24 hours.

Compound (XV) and Compound (XVI) employed here may be commercially available compounds or can be synthesized by a method known in the art.

Next, a reaction for conducting a nucleophilic replacement reaction of the carbon atom to which an alkoxycarbonyl group is bound in Compound (XIII) onto Compound (XIV) to produce a carbon-carbon bond thereby obtaining Compound (XII) is described.

This reaction is usually conducted in a solvent in the presence of a base.

The amount of Compound (XIV) per mole of Compound (XIII) is usually 0.5 to 10 moles, preferably 0.8 to 5 moles.

The base employed may be, for example, but is not limited to, alkaline metal hydrides such as sodium hydride, alkaline metal carbonates such as sodium carbonate and potassium carbonate. The amount of the base employed per mole of Compound (XIII) is usually 0.5 to 10 moles, preferably 0.8 to 5 moles.

Since, in the reaction for obtaining Compound (XIII) in the presence of a base from Compound (XV) described above, the acidity of the hydrogen atom of the methylene group between the carbonyl group and the ester group of the resultant Compound (XIII) is higher than the acidity of the hydrogen atom of the acetyl group of Compound (XV), an alkaline metal salt and the like of Compound (XIII) is formed during the course of the reaction, thus allowing the reaction solution of Compound (XIII) to be employed directly without isolation. In such a case, the reaction can be conducted without any particular additional use of the base.

The reaction temperature is usually 0 degrees C. to 250 degrees C., preferably room temperature to 150 degrees C., and the reaction time is usually 0.1 hour to several days, preferably 0.5 hour to 24 hours.

Consecutively, a reaction for obtaining Compound (XI) by hydrolysis/decarbonation of Compound (XII) is described.

This hydrolysis/decarbonation reaction can be conducted in a solvent under both of a basic condition and an acidic condition.

When conducting under the basic condition, the base is usually an alkaline metal salt base such as sodium hydroxide and potassium hydroxide. The solvent is usually water, as well as water combined with alcohols.

When conducting under the acidic condition, the acid catalyst is preferably an inorganic acid such as hydrochloric acid, hydrobromic acid, and sulfuric acid, as well as an organic acid such as acetic acid. The solvent is usually water, or water combined with an organic acid such as acetic acid.

The reaction temperature is usually 0 degrees C. to reflux temperature, preferably 10 degrees C. to reflux temperature. The reaction time is usually 0.1 hour to several days, preferably 0.5 hour to 24 hours.

A method which can otherwise be employed involves conducting hydrolysis first under a basic condition followed by decarbonation under an acidic condition, or heating a beta-keto carboxylic acid obtained in the hydrolysis in an organic solvent thereby accomplishing decarbonation. In such cases, the bases and the acids employed are those listed above.

Finally, a reaction for obtaining Compound (VIII-a) by converting Compound (XI) into an oxirane.

This reaction may involve reacting Compound (XI) with a sulfur ylide including sulfonium methylides such as dimethylsulfonium methylide or sulfoxonium methylides such as dimethyl sulfoxonium methylide in a solvent.

The sulfonium methylides and the sulfoxonium methylides employed can be produced by reacting, in a solvent, a sulfonium salt (for example, trimethylsulfonium iodide, and trimethylsulfonium bromide) or a sulfoxonium salt (for example, trimethylsulfoxonium iodide and trimethylsulfoxonium bromide) with a base.

The amount of such a sulfonium methylide and sulfoxonium methylide per mole of Compound (XI) is 0.5 to 10 moles, preferably 0.8 to 5 moles.

While the solvent employed is not limited particularly, those which may be exemplified include aromatic hydrocarbons such as toluene and xylene, amides such as N,N-dimethylformamide, N,N-dimethylacetamide, and N-methyl-2-pyrrolidinone, ethers such as diethyl ether, tetrahydrofuran, and dioxane, as well as dimethyl sulfoxide. Two or more of these solvents may be employed in combination.

When water is used in the reaction, it may be used as being admixed with an organic solvent, and, when used together with a hydrophobic organic solvent, a phase transfer catalyst such as quaternary ammonium salts, crown ether and its analogues may be added if desired to the reaction mixture thereby effecting the reaction. The quaternary ammonium salts may be, for example, tetrabutylammonium salt, trimethylbenzylammonium salt and triethylbenzylammonium salt.

When using an alkaline metal hydroxide such as sodium hydroxide, and potassium hydroxide in an organic solvent such as toluene, it may sometimes be preferable to add alcohols such as diethylene glycol.

In such a case, the amount of the alcohol employed per mole of Compound (XI) is usually 0.001 moles to 10 moles, preferably 0.005 to 5 moles.

While the base employed for producing sulfonium methylides and sulfoxonium methylides are not limited particularly, those employed preferably include an alkaline metal hydroxide such as sodium hydroxide, and potassium hydroxide, a metal hydride such as sodium hydride, an alkoxide of an alkaline metal such as sodium methoxide, sodium ethoxide, sodium t-butoxide, and potassium t-butoxide.

The reaction temperature and the reaction time may appropriately be selected depending on the types of the solvent, Compound (XI), sulfonium salt or sulfoxonium salt, base and the like which are employed. The reaction temperature is preferably −100 degrees C. to 200 degrees C., more preferably −50 degrees C. to 150 degrees C. The reaction time is preferably 0.1 hour to several days, more preferably 0.5 hour to 2 days.

(4) Second Method for Producing Compound (I)

(4-1) Step B1

Another embodiment of the production method according to the invention comprises a step for reacting Compound (IV) described above with a carbonyl compound represented by Formula (V) shown below (hereinafter referred to as “Compound (V)”) (Step B1) (see Scheme (8) shown below).

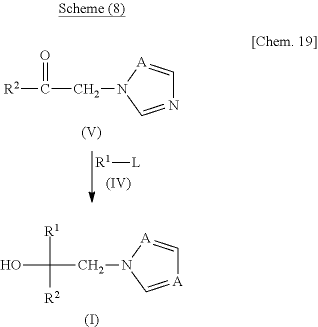

Herein, the contexts of the definitions of R¹, R², A, and L are as defined above. In this step, an alkaline earth metal-Q (Q is a halogen atom) is employed more preferably as L.

In this step, a carbon-carbon bond is formed by a nucleophilic addition reaction by Compound (IV) onto the carbonyl carbon atom of Compound (V).

While the solvent employed is not limited particularly as long as it is an inert solvent, it may be, for example, ethers such as diethyl ether, tetrahydrofuran, and dioxane, aromatic hydrocarbons such as benzene, toluene, and xylene.

The amount of Compound (IV) employed per mole of Compound (V) is usually 0.5 to 10 moles, preferably 0.8 to 2 moles. Compound (IV) prepared immediately before use is preferred. It is also possible to add a Lewis acid if desired, and in such a case the amount of the base employed per mole of Compound (IV) is usually more than 0 and not more than 5 moles, preferably 0.1 to 1 moles. The Lewis acid employed may be, for example, aluminum chloride, zinc chloride, and cerium chloride.

The reaction temperature and the reaction time may appropriately be selected depending on the types of the solvent, Compound (V) and Compound (IV) and the like which are employed. The reaction temperature is preferably −100 degrees C. to 100 degrees C., more preferably −70 degrees C. to 50 degrees C. The reaction time is preferably 0.1 to 12 hours, more preferably 0.5 to 6 hours.

(4-2) Step B2

Compound (V) employed in Step B1 can be obtained by a known method (for example, see Japanese Unexamined Patent Application Publication No. 64-22857). Compound (V) can be obtained also by the reaction for example of Compound (VII) and Compound (III) described above (see Scheme (9) shown below).

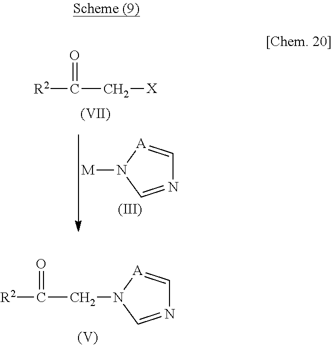

Herein, the contexts of the definitions of R², A, X and M are as defined above.

3. Agro-Horticultural Agents and Industrial Material Protecting Agents

The utilities of an azole derivative according to the invention (Compound (I)) as an agro-horticultural agent and an industrial material protecting agent (hereinafter also referred to as “agro-horticultural agent and the like”) are described below.

Since Compound (I) has a 1,2,4-triazolyl group or an imidazolyl group, it forms an acid addition salt of an inorganic acid or an organic acid, as well as a metal complex. Accordingly, it can be employed also in the form of a moiety of the acid addition salt or the metal complex as an active ingredient of an agro-horticultural agent and the like.

Furthermore, Compound (I) may have one or more asymmetric carbon atoms depending on the structures represented by R¹ and R². Therefore, depending on the composition, it may be a stereoisomer mixture, an optical isomer mixture, either stereoisomer, or either optical isomer. Accordingly, at least one of these stereoisomers or optical isomers can be employed also as an active ingredient of an agro-horticultural agent and the like.

(1) Plant Disease Controlling Effects

Compound (I) of the invention exhibits a controlling effect on a broad range of plant diseases. Applicable diseases are exemplified below.

Soybean rust (Phakopsora pachyrhizi, Phakopsora meibomiae), rice blast (Pyricularia grisea), rice brown spot (Cochliobolus miyabeanus), rice leaf blight (Xanthomonas oryzae), rice sheath blight (Rhizoctonia solani), rice stem rot (Helminthosporium sigmoideun), rice bakanae disease (Gibberella fujikuroi), rice bacterial seedling blight (Pythium aphanidermatum), apple powdery mildew (Podosphaera leucotricha), apple scab (Venturia inaequalis), apple blossom blight (Monilinia mali), apple alternaria blotch (Alternaria alternata), apple valsa canker (Valsa mali), pear black spot (Alternaria kikuchiana), pear powdery mildew (Phyllactinia pyri), pear rust (Gymnosporangium asiaticum), pear scab (Venturia nashicola), grape powdery mildew (Uncinula necator), grape downy mildew (Plasmopara viticola), grape ripe rot (Glomerella cingulata), barley powdery mildew (Erysiphe graminis f. sp hordei), barley stem rust (Puccinia graminis), barley stripe rust (Puccinia striiformis), barley stripe (Pyrenophora graminea), barley leaf blotch (Rhynchosporium secalis), wheat powdery mildew (Erysiphe graminis f. sp tritici), wheat leaf rust (Puccinia recondita), wheat stripe rust (Puccinia striiformis), wheat eye spot (Pseudocercosporella herpotrichoides), wheat fusarium blight (Fusarium graminearum, Microdochium nivale), wheat glume blotch (Phaeosphaeria nodorum), wheat leaf blight (Septoria tritici), gourd powdery mildew (Sphaerotheca fuliginea), gourd anthracnose (Colletotrichum lagenarium), cucumber downy mildew (Pseudoperonospora cubensis), cucumber Phytophthora rot (Phytophthora capsici), tomato powdery mildew (Erysiphe cichoracearum), tomato early blight (Alternaria solani), eggplant powdery mildew (Erysiphe cichoracearum), strawberry powdery mildew (Sphaerotheca humuli), tobacco powdery mildew (Erysiphe cichoracearum), sugar beet cercpspora leaf spot (Cercospora beticola), maize smut (Ustillaga maydis), plum brown rot (Monilinia fructicola), various plants-affecting gray mold (Botrytis cinerea), sclerotinia rot (Sclerotinia sclerotiorum) and the like may be exemplified.

In addition, grape rust (Phakopsora ampelopsidis), watermelon wilt (Fusarium oxysporum f.sp. niveum), cucumber wilt (Fusarim oxysporum f.sp. cucumerinum), white radish yellow (Fusarium oxysporum f.sp. raphani), tobacco brown spot (Alternaria longipes), potato early blight (Alternaria solani), soybean brown spot (Septoria glycines), soybean purple stain (Cercospora kikuchii) and the like may be exemplified.

Examples of applicable plants may be, for example, wild plants, cultivated plant varieties, plants and cultivated plant varieties obtained by conventional biological breeding such as heterologous mating or plasma fusion, and plants and cultivated plant varieties obtained by genetic engineering. The genetically-engineered plants and the cultivated plant varieties may be, for example, herbicide-resistant crops, vermin-resistant crops having insecticidal protein-producing genes integrated therein, disease-resistant crops having disease resistance inducer-producing genes integrated therein, palatably improved crops, productively improved crops, preservably improved crops, and productively improved crops. The genetically-engineered cultivated plant varieties may be, for example, those involving trade marks such as ROUNDUP READY, LIBERTY LINK, CLEARFIELD, YIELDGARD, HERCULEX, BOLLGARD and the like.

(2) Plant Growth Promoting Effect

Furthermore, on a broad range of crops and horticultural plants, Compound (I) exhibits yield-increasing effects by regulating the growth of the crops and plants, or quality-improving effects. Such crops may be, for example, those listed below.

Wheat, barley, oats, rice, rapeseed, sugarcane, corn, maize, soybean, pea, peanut, sugar beet, cabbage, garlic, radish, carrot, apple, pear, citric fruits such as mandarin, orange, and lemon, peach, cherry, avocado, mango, papaya, red pepper, cucumber, melon, strawberry, tobacco, tomato, eggplant, lawn grass, chrysanthemum, azalea, and other ornamental plants.

(3) Industrial Material Protecting Effect

Moreover, Compound (I) exhibits an excellent ability of protecting an industrial material from a broad spectrum of hazardous microorganisms which invade such a material. Examples of such microorganisms are listed below.

Paper/pulp deteriorating microorganisms (including slime-forming microorganisms) such as Aspergillus sp., Trichoderma sp., Penicillium sp., Geotrichum sp., Chaetomium sp., Cadophora sp., Ceratostomella sp., Cladosporium sp., Corticium sp., Lentinus sp., Lenzites sp., Phoma sp., Polysticus sp., Pullularia sp., Stereum sp., Trichosporium sp., Aerobacter sp., Bacillus sp., Desulfovibrio sp., Pseudomonas sp., Flavobacterium sp., and Micrococcus sp.; fiber-deteriorating microorganisms such as Aspergillus sp., Penicillium sp., Chaetomium sp., Myrothecium sp., Curvularia sp., Gliomastix sp., Memnoniella sp., Sarcopodium sp., Stachybotrys sp., Stemphylium sp., Zygorhynchus sp., Bacillus sp. and Staphylococcus sp.; lumber-deteriorating microorganisms such as Tyromyces palustris, Coriolus versicolor, Aspergillus sp., Penicillium sp., Rhizopus sp., Aureobasidium sp., Gliocladium sp., Cladosporium sp., Chaetomium sp., and Trichoderma sp.; leather-deteriorating microorganisms such as Aspergillus sp., Penicillium sp., Chaetomium sp., Cladosporium sp., Mucor sp., Paecilomyces sp., Pilobus sp., Pullularia sp., Trichosporon sp., and Tricothecium sp.; rubber/plastic-deteriorating microorganisms such as Aspergillus sp., Penicillium sp., Rhizopus sp., Trichoderma sp., Chaetomium sp., Myrothecium sp., Streptomyces sp., Pseudomonas sp., Bacillus sp., Micrococcus sp., Serratia sp., Margarinomyces sp., and Monascus sp.; paint-deteriorating microorganisms such as Aspergillus sp., Penicillium sp., Cladosporium sp., Aureobasidium sp., Gliocladium sp., Botryodiplodia sp., Macrosporium sp., Monilia sp., Phoma sp., Pullularia sp., Sporotrichum sp., Trichoderma sp., Bacillus sp., Proteus sp., Pseudomonas sp., and Serratia sp.

(4) Formulations

While Compound (I) may be applied, as an active ingredient of an agro-horticultural agent, alone without any other components, it is usually combined with a solid carrier, a liquid carrier, a surfactant, or other formulation auxiliary agents to be formulated into various formulations such as a powder, wettable powder, granule, and emulsifiable concentrate.

Such a formulation is formulated so that it contains Compound (I) as an active ingredient in an amount of 0.1 to 95% by weight, preferably 0.5 to 90% by weight, more preferably 2 to 80% by weight.

Examples of carriers, diluents and surfactants employed as formulation auxiliary agents are solid carriers including talc, kaolin, bentonite, diatomaceous earth, white carbon, and clay. The liquid diluents include water, xylene, toluene, chlorobenzene, cyclohexane, cyclohexanone, dimethyl sulfoxide, dimethyl formamide, and alcohols. The surfactant may be appropriately selected for an intended effect, and the emulsifier may be, for example, polyoxyethylene alkylaryl ether, polyoxyethylene sorbitan monolaurate. The dispersing agent may be, for example, lignin sulfonate, and dibutylnaphthalene sulfonate, and the wetting agent may be, for example, an alkyl sulfonate and alkylphenyl sulfonate.

The formulation may be used as it is, or used as being diluted in a diluent such as water to a certain concentration. The concentration of Compound (I) when used as being diluted is preferably 0.001 to 1.0%.

The amount of Compound (I) for 1 ha of the agro-horticultural field such as a farm, paddy field, orchard, and greenhouse is 20 to 5000 g, more preferably 50 to 2000 g. Since these concentration and amount to be used may vary depending on the dosage form, timing of use, method of use, place of use, subject crop and the like, they can be increased or decreased regardless of the ranges mentioned above.

In addition, Compound (I) can be combined with other active ingredients, including bactericides, insecticides, acaricides, and herbicides, such as those listed below, thereby enabling the use as an agro-horticultural agent having an enhanced performance.

<Anti-Bacterial Substances>

Acibenzolar-S-methyl, 2-phenylphenol (OPP), azaconazole, azoxystrobin, amisulbrom, bixafen, benalaxyl, benomyl, benthiavalicarb-isopropyl, bicarbonate, biphenyl, bitertanol, blasticidin-S, borax, Bordeaux mixture, boscalid, bromuconazole, bronopol, bupirimate, sec-butylamine, calcium polysulphide, captafol, captan, carbendazim, carboxin, carpropamid, quinomethionate, chloroneb, chloropicrin, chlorothalonil, chlozolinate, cyazofamid, cyflufenamid, cymoxanil, cyproconazole, cyprodinil, dazomet, debacarb, dichlofluanid, diclocymet, diclomezine, dicloran, diethofencarb, difenoconazole, diflumetorim, dimethomorph, dimethoxystrobin, diniconazole, dinocap, diphenylamine, dithianon, dodemorph, dodine, edifenphos, epoxiconazole, ethaboxam, ethoxyquin, etridiazole, enestroburin, famoxadone, fenamidone, fenarimol, fenbuconazole, fenfuram, fenhexamid, fenoxanil, fenpiclonil, fenpropidin, fenpropimorph, fentin, ferbam, ferimzone, fluazinam, fludioxonil, flumorph, fluoroimide, fluoxastrobin, fluquinconazole, flusilazole, flusulfamide, flutolanil, flutriafol, folpet, fosetyl-Al, fuberidazole, furalaxyl, furametpyr, fluopicolide, fluopyram, guazatine, hexachlorobenzene, hexaconazole, hymexazol, imazalil, imibenconazole, iminoctadine, ipconazole, iprobenfos, iprodione, iprovalicarb, isoprothiolane, isopyrazam, isotianil, kasugamycin, copper preparations, such as: copper hydroxide, copper naphthenate, copper oxychloride, copper sulphate, copper oxide, oxine copper, kresoxim-methyl, mancopper, mancozeb, maneb, mandipropamid, mepanipyrim, mepronil, metalaxyl, metconazole, metiram, metominostrobin, mildiomycin, myclobutanil, nitrothal-isopropyl, nuarimol, ofurace, oxadixyl, oxolinic acid, oxpoconazole, oxycarboxin, oxytetracycline, pefurazoate, orysastrobin, penconazole, pencycuron, penthiopyrad, pyribencarb, fthalide, picoxystrobin, piperalin, polyoxin, probenazole, prochloraz, procymidone, propamocarb, propiconazole, propineb, proquinazid, prothioconazole, pyraclostrobin, pyrazophos, pyrifenox, pyrimethanil, pyroquilon, quinoxyfen, quintozene, silthiopham, simeconazole, spiroxamine, Sulfur and sulfur formulations, tebuconazole, tecloftalam, tecnazen, tetraconazole, thiabendazole, thifluzamide, thiophanate-methyl, thiram, thiadinil, tolclofosmethyl, tolylfluanid, triadimefon, triadimenol, triazoxide, tricyclazole, tridemorph, trifloxystrobin, triflumizole, triforine, triticonazole, validamycin, vinclozolin, zineb, ziram, zoxamide, amisulbrom, sedaxane, flutianil, valiphenal, ametoctradin, dimoxystrobin, metrafenone, hydroxyisoxazole, metasulfocarb and the like.

<Insecticides/Acaricides/Nematocides>

Abamectin, acephate, acrinathrin, alanycarb, aldicarb, allethrin, amitraz, avermectin, azadirachtin, azamethiphos, azinphos-ethyl, azinphos-methyl, azocyclotin, Bacillus firmus, Bacillus subtilis, Bacillus thuringiensis, bendiocarb, benfuracarb, bensultap, benzoximate, bifenazate, bifenthrin, bioallethrin, bioresmethrin, bistrifluoron, buprofezin, butocarboxim, butoxycarboxim, cadusafos, carbaryl, carbofuran, carbosulfan, cartap, CGA50439, chlordane, chlorethoxyfos, chlorphenapyr, chlorfenvinphos, chlorfluazuron, chlormephos, chlorpyrifos, chlorpyrifos methyl, chromafenozide, clofentezine, clothianidin, chlorantraniliprole, coumaphos, cryolite, cyanophos, cycloprothrin, cyfluthrin, cyhalothrin, cyhexatin, cypermethrin, cyphenothrin, cyromazine, Cyazapyr, cyenopyrafen, DCIP, DDT, deltamethrin, demeton-S-methyl, diafenthiuron, diazinon, dichlorophen, dichloropropene, dichlorvos, dicofol, dicrotophos, dicyclanil, diflubenzuron, dimethoate, dimethylvinphos, dinobuton, dinotefuran, emamectin, endosulfan, EPN, esfenvalerate, ethiofencarb, ethion, ethiprole, ethofenprox, ethoprophos, etoxazole, famphur, fenamiphos, fenazaquin, fenbutatin oxide, fenitrothion, fenobucarb, fenothiocarb, fenoxycarb, fenpropathrin, fenpyroximate, fenthion, fenvalerate, fipronil, flonicamid, fluacrypyrim, flucycloxuron, flucythrinate, flufenoxuron, flumethrin, fluvalinate, flubendiamide, formetanate, fosthiazate, halfenprox, furathiocarb, halofenozide, gamma-HCH, heptenophos, hexaflumuron, hexythiazox, hydramethylnon, imidacloprid, imiprothrin, indoxacarb, isoprocarb, isoxathion, lufenuron, malathion, mecarbam, metam, methamidophos, methidathion, methiocarb, methomyl, methoprene, methothrin, methoxyfenozide, metolcarb, milbemectin, monocrotophos, naled, nicotine, nitenpyram, novaluron, noviflumuron, omethoate, oxamyl, oxydemethon methyl, parathion, permethrin, phenthoate, phorate, phosalone, phosmet, phosphamidon, phoxim, pirimicarb, pirimiphos-methyl, profenofos, propoxur, prothiophos, pymetrozin, pyrachlophos, pyrethrin, pyridaben, pyridalyl, pyrimidifen, pyriproxifen, pyrifluquinazon, pyriprole, quinalphos, silafluofen, spinosad, spirodiclofen, spiromesifen, spirotetramat, sulfluramid, sulphotep, SZI-121, tebufenozid, tebufenpyrad, tebupirimphos, teflubenzuron, tefluthrin, temephos, terbufos, tetrachlorvinphos, thiacloprid, thiamethoxam, thiodicarb, thiofanox, thiometon, tolfenpyrad, tralomethrin, tralopyril, triazamate, triazophos, trichlorfon, triflumuron, vamidothion, valifenal, XMC, xylylcarb, imicyafos, lepimectin and the like.

<Plant Growth Regulators>

Ancymidol, 6-benzylaminopurine, paclobutrazol, diclobutrazole, uniconazole, methylcyclopropene, mepiquat chloride, ethefon, chlormequat chloride, inabenfide, prohexadione and its salts, trinexapac-ethyl and the like. As plant hormones, jasmonic acid, bras sinosteroid, gibberellin and the like.

While Compound (I) may be applied, as an active ingredient of an industrial material protecting agent, alone without any other components, it is generally dissolved or dispersed in a suitable liquid carrier, or mixed with a solid carrier, and combined if necessary with emulsifier, dispersing agent, spreading agent, penetrating agent, wetting agent, stabilizer and the like and formulated into a dosage form such as wettable powder, powder, granule, tablet, paste, suspension, and spray. It may also be supplemented with other bactericides, insecticides, deterioration-preventing agent and the like.

The liquid carrier may be any liquid as long as it does not react with an active ingredient, and may be selected from water, alcohols (for example, methyl alcohol, ethyl alcohol, ethylene glycol, and cellosolve), ketones (for example, acetone and methylethylketone), ethers (for example, dimethyl ether, diethyl ether, dioxane, and tetrahydrofuran), aromatic hydrocarbons (for example, benzene, toluene, xylene, and methylnaphthalene), aliphatic hydrocarbons (for example, gasoline, kerosene, paraffin oil, machine oil, and fuel oil), acid amides (for example, dimethyl formamide and N-methylpyrrolidone), halogenated hydrocarbons (for example, chloroform and carbon tetrachloride), esters (for example, acetic acid ethyl ester and fatty acid glycerin ester), nitriles (for example, acetonitrile), and dimethyl sulfoxide and the like.

The solid carrier may be, for example, a microparticle or a granule of kaolin clay, bentonite, acid clay, pyrophylite, talc, diatomaceous earth, calcite, urea, and ammonium sulfate.

The emulsifiers and the dispersing agents may be, for example, soaps, alkyl sulfonates, alkylaryl sulfonates, dialkyl sulfosuccinates, quaternary ammonium salts, oxyalkylamines, fatty acid esters, polyalkylene oxide-based, anhydrosorbitol-based surfactants.

When Compound (I) is contained as an active ingredient in a formulation, it is added generally in such an amount that the concentration becomes 0.1 to 99.9% by weight, although the content may vary depending on the dosage form and the purpose of use. Upon being used practically, it is combined appropriately with a solvent, diluent, extender and the like so that the treatment concentration is usually 0.005 to 5% by weight, preferably 0.01 to 1% by weight.

As described above, an azole derivative represented by Compound (I) exhibits an excellent biocidal effect on a large number of microorganisms which induce diseases in plants. That is, by incorporating the azole derivative represented by Compound (I) as an active ingredient, an agro-horticultural disease controlling agent having a low toxicity to humans and animals, capable of being handled safely, and exhibiting a high controlling effect on a wide range of plant diseases can be realized.

(Remarks)

The invention is not limited to the embodiments described above, and it may be varied in various ways within the scope of the appended Claims. That is, an embodiment achieved by combining technical means varied appropriately within the scope of the appended Claims will be included in the technical scope of the invention.

### EXAMPLES

The invention is embodied below with referring to Production Examples, Formulation Examples, and Experimental Examples. The invention is not restricted to the following Production Examples, Formulation Examples, and Experimental Examples unless departing from its scope.

When 2 or more asymmetric carbon atoms are present in Compound (I), a plurality of diastereomers as isomers are formed. It is difficult to separate and assign all of these diastereomers. Accordingly, in the following Production Examples and the like, only diastereomers which could be assigned are indicated in alphabetical order. The order of this alphabetical order has no particular meaning, and just the order of the assignment is indicated, such as Compound I-2a and Compound I-2b.

### Production Example 1

### Synthesis of 1-(1-chlorocyclopropyl)-1-(2,2-dichlorocyclopropyl)-2-(1H-1,2,4-triazol-1-yl)ethanol (Compound No. I-2)

### (1) Synthesis of intermediate 1-chloro-2-(1-chlorocyclopropyl)-3-buten-2-ol (Compound (IX), R²=1-chlorocyclopropyl, R⁸=H, R⁹=H, R¹⁰=H, X=Cl, n=0)

Under nitrogen flow, 2-chloro-1-(1-chlorocyclopropyl)ethanone (Compound (VII), R²=1-chlorocyclopropyl, X=Cl) (0.67 g, 4.38 mmol) in anhydrous THF (5 ml) was cooled to −20 degrees C. To this solution, a solution of 0.75M vinyl magnesium bromide (Compound (X), R⁸=H, R⁹=H, R¹⁰=H, L=MgBr, n=0) (12.5 ml, 9.38 mmol) diluted in anhydrous THF (6 ml) was added dropwise in such a manner that the reaction temperature was not elevated. After completion of the addition, followed by warming slowly to 0 degrees C., stirring was conducted for 1 hour at 0 degrees C. After cooling with ice/water, the reaction solution was combined with a saturated ammonium chloride solution, and extracted with diethyl ether. The organic layer was washed with saturated sodium bicarbonate, water, saturated brine, and then dried over anhydrous sodium sulfate, and then the solvent was distilled away. The resultant oil was purified by silica gel column chromatography (eluent (hexane-ethyl acetate=20:1)) to obtain the desired substance.

Product: 0.53 g

Yield: 67%

Description: Colorless oil

NMR delta_{H} (400 MHz, CDCl₃):

0.93-1.04 (m, 2H), 1.07-1.12 (m, 1H), 1.24-1.30 (m, 1H), 2.34 (s, 1H), 3.90 3.93 (d×2, 2H, J=11.3 Hz), 5.36 (dd, 1H, J=0.8, 10.8 Hz), 5.51 (dd, 1H, J=0.7, 17.2 Hz), 6.05 (dd, 1H, J=10.8, 17.2 Hz).

### (2) Synthesis of Intermediate 2-(1-chlorocyclopropyl)-2-(2,2-dichlorocyclopropyl)oxirane (Compound (II-a), R²=1-chlorocyclopropyl, R⁴=H, R⁵=H, R⁶=H, X¹=Cl, X²=Cl, n=0)

1-chloro-2-(1-chlorocyclopropyl)-3-buten-2-ol (Compound (IX), R²=1-chlorocyclopropyl, R⁸=H, R⁹=H, R¹⁰=H, X=Cl, n=0) (0.53 g, 2.93 mmol) was dissolved in chloroform (2.4 ml) and benzyltriethylammonium chloride (34 mg, 0.15 mmol) was added. To this solution, a solution of sodium hydroxide (1.80 g, 45.0 mmol) dissolved in water (1.8 ml) was added, and vigorous stirring was conducted for 8 hours at 60 degrees C. Thereafter, the reaction temperature was adjusted to 70 degrees C. for further 4 hours, and stirring was conducted for 20 hours at a reaction temperature of 80 degrees C. After the reaction, extraction was made with chloroform, and the organic layer was washed with water and saturated brine, and then dried over anhydrous sodium sulfate. The solvent was distilled away under reduced pressure, and the resultant oil was purified by silica gel column chromatography (eluent (hexane-ethyl acetate=20:1)) to obtain the desired substance as respective 2 isomers.

<Isomer a>

Product: 41 mg

Yield: 6.3%

Description: Pale yellow oil

NMR delta_{H} (400 MHz, CDCl₃):

0.9-0.97 (m, 2H), 1.19-1.25 (m, 2H), 1.46-1.52 (m, 1H), 1.54, 1.56 (d×2, 1H, J=10.9 Hz), 2.54 (d, 1H, J=5.5 Hz), 2.68 (dd, 1H, J=8.4, 10.8 Hz), 2.75 (d, 1H, J=5.5 Hz).

<Isomer b>

Product: 37 mg

Yield: 5.7%

Description: Pale yellow oil

NMR delta_{H} (400 MHz, CDCl₃):

0.99-1.11 (m, 2H), 1.13-1.26 (m, 2H), 1.28-1.38 (m, 1H), 1.66 (dd, 1H, J=7.8, 11.0 Hz), 2.40 (dd, 1H, J=8.3, 11.0 Hz), 2.76 (d, 1H, J=3.6 Hz), 2.83 (dd, 1H, J=0.7, 3.9 Hz).

### (3) Synthesis of 1-(1-chlorocyclopropyl)-1-(2,2-dichlorocyclopropyl)-2-(1H-1,2,4-triazol-1-yl)ethanol Compound No. I-2a)

Under nitrogen flow, 1H-1,2,4-triazole (Compound (III), M=H) (15 mg, 0.22 mmol), potassium carbonate (31 mg, 0.22 mmol) and potassium t-butoxide (1.7 mg, 0.02 mmol) were suspended in NMP (2 ml). A solution of 2-(1-chlorocyclopropyl)-2-(2,2-dichlorocyclopropyl)oxirane (a, one of the isomers of Compound (II-a), R²=1-chlorocyclopropyl, R⁸=H, R⁹=H, R¹⁰=H, X¹=Cl, X²=Cl, n=0) (37 mg, 0.16 mmol) in NMP (1 ml) was added and stirring was conducted for 5 hours at 80 degrees C. The reaction solution was poured into ice/water, and extracted with ethyl acetate. The organic layer was washed with water and saturated brine, and then dried over anhydrous sodium sulfate. The solvent was distilled away under reduced pressure, and the resultant crude product was purified by silica gel column chromatography (eluent (hexane-ethyl acetate=1:1)) to obtain the desired substance.

Product: 12 mg

Yield: 25%

Description: White crystal, Melting point: 70 to 71 degrees C.

NMR delta_{H} (400 MHz, CDCl₃):

0.74-0.87 (m, 2H), 1.09-1.15 (m, 1H), 1.35-1.41 (m, 2H), 1.52 (dd, J=11.0, 7.1 Hz, 1H), 2.20 (dd, J=11.0, 9.1 Hz, 1H), 3.75 (s, 1H), 4.51 (d, J=14.2 Hz, 1H), 4.60 (d, J=14.2 Hz, 1H), 7.98 (s, 1H), 8.22 (s, 1H).

### Production Example 2

### Synthesis of 2-(1-chlorocyclopropyl)-1-(2,2-dibromocyclopropyl)-3-(1H-1,2,4-triazol-1-yl)propan-2-ol (Compound No. I-210)

### (1) Synthesis of intermediate 1-chloro-2-(1-chlorocyclopropyl)-4-penten-2-ol (Compound (IX), R²=1-chlorocyclopropyl, R⁸=H, R⁹=H, R¹⁰=H, R¹¹=H, R¹²=H, X=Cl, n=1)

Under argon atmosphere, 2-chloro-1-(1-chlorocyclopropyl)ethanone (Compound (VII), R²=1-chlorocyclopropyl, X=Cl) (1.5 g, 0.0098 mol) was dissolved in diethyl ether (20 ml), and cooled to about −50 degrees C. 1M Diethyl ether solution of allyl-magnesium bromide (Compound (X), R⁸=H, R⁹=H, R¹⁰=H, R¹¹=H, R¹²=H, L=MgBr, n=1) (18 ml, 0.0098×1.8 mol) was added, and stirred at the same temperature for about 20 minutes, and then temperature was elevated slowly while stirring for 1 hour. After stirring for further 1 hour under cooling with ice, ice/water and a saturated aqueous solution of ammonium chloride were added. After extraction with diethyl ether, the organic layer was extracted with saturated sodium bicarbonate and saturated brine, dried over anhydrous sodium sulfate, and then concentrated to obtain the desired substance as a crude material.

Crude product: 1.48 g

Crude yield: 77%

Description: Colorless oil

NMR delta_{H} (400 MHz, CDCl₃):

0.9-1.0 (m, 2H), 1.1-1.2 (m, 1H), 1.2-1.3 (m, 1H), 2.13 (s, 1H), 2.57 (dd, J=14.3, 8.4 Hz, 1H), 2.70 (ddt, J=14.3, 6.5, 1.3 Hz, 1H), 3.83 (d, J=11.4 Hz, 1H), 3.95 (d, J=11.4 Hz, 1H), 5.1-5.2 (m, 1H), 5.22 (bs, 1H), 5.9-6.1 (m, 1H).

### (2) Synthesis of intermediate 2-(1-chlorocyclopropyl)-2-(2,2-dibromocyclopropylmethyl)oxirane (Compound (II-a), R²=1-chlorocyclopropyl, R⁸=H, R⁹=H, R¹⁰=H, R¹¹=H, R¹²=H, X¹=Br, X²=Br, n=1)

Crude 1-chloro-2-(1-chlorocyclopropyl)-4-penten-2-ol (Compound (IX), R²=1-chlorocyclopropyl, R⁸=H, R⁹=H, R¹⁰=H, R¹¹=H, R¹²=H, X=Cl, n=1) (0.60 g, 0.0031 mol) was combined with bromoform (2.33 g, 9.2 mmol), 50% aqueous solution of sodium hydroxide (2 g), and benzyltriethylammonium chloride (35 mg, 0.154 mmol) and stirred at room temperature for 1 hour, at about 60 degrees C. for 1 hour, and then at about 80 degrees C. for 1 hour. The reaction solution was combined with water and extracted with diethyl ether. The organic layer was washed with water and saturated brine, and then dried over anhydrous sodium sulfate and concentrated. The resultant crude product was combined with bromoform (2.33 g, 9.2 mmol), 50% aqueous solution of sodium hydroxide (2 g), and benzyltriethylammonium chloride (70 mg, 0.30 mol) and stirred at about 80 degrees C. for 4 hours. The reaction solution was combined with water and extracted with diethyl ether. The organic layer was washed with water and saturated brine, and then dried over anhydrous sodium sulfate and concentrated. Purification by silica gel column chromatography (eluent (hexane-ethyl acetate=20:1)) gave a crude product which was used as it was in the next reaction.

Crude product: 0.60 g

Crude yield: 59%

Description: Oil

### (3) Synthesis of 2-(1-chlorocyclopropyl)-1-(2,2-dibromocyclopropyl)-3-(1H-1,2,4-triazol-1-yl)propan-2-ol (Compound No. I-210)

Potassium carbonate (0.38 g, 2.7 mmol) was suspended in DMF (3 ml), and then t-BuONa (0.035 g, 0.36 mmol) and 1,2,4-triazole (Compound (III), M=H) (0.19 g, 2.7 mmol) were added. Crude 2-(1-chlorocyclopropyl)-2-(2,2-dibromocyclopropylmethyl)oxirane (Compound (II-a), R²=1-chlorocyclopropyl, R⁸=H, R⁹=H, R¹⁰=H, R¹¹=H, R¹²=H, X¹=Br, X²=Br, n=1) (0.60 g, 0.0018 mol) dissolved in DMF (3 ml) was added and stirring was conducted at about 90 degrees C. for 2 hours. Ethyl acetate and water were added, and then after partitioning the organic layer was washed with saturated brine. The aqueous layer was extracted with ethyl acetate, and then the organic layer was dried over anhydrous sodium sulfate and concentrated. Purification was conducted by silica gel column chromatography (eluent (hexane-ethyl acetate=1:1)), and Isomer a having a lower polarity among two isomers was isolated.

<Compound No. I-210a>

Product: 0.065 g

Yield: 9%

Description: White solid, Melting point: 114 degrees C.

NMR delta_{H} (400 MHz, CDCl₃):

0.28-0.38 (m, 1H), 0.42-0.52 (m, 1H), 0.73-0.84 (m, 1H), 1.02-1.12 (m, 1H), 1.42 (app.t, J=7.6 Hz, 1H), 1.88 (dd, J=7.3, 10.6 Hz, 1H), 1.92-2.19 (m, 3H), 4.36 (s, 1H), 4.39 (d, J=14.2 Hz, 1H), 4.95 (d, J=14.2 Hz, 1H), 8.04 (s, 1H), 8.28 (s, 1H).

### Production Example 3

### Synthesis of 1,3-bis(2,2-dichlorocyclopropyl)-2-(1H-1,2,4-triazol-1-ylmethyl)propan-2-ol (Compound No. I-277)

### (1) Synthesis of intermediate 4-chloromethylhepta-1,6-dien-4-ol

Under nitrogen flow, magnesium (0.58 g, 24 mmol) was combined with anhydrous diethyl ether (10 ml), and then treated dropwise with a solution of allyl bromide (2.70 g, 22.3 mmol) dissolved in diethyl ether (25 ml) in such a manner that the reaction solution kept refluxing gently, and then stirred at room temperature for 30 minutes. A solution of chloroacetyl chloride (1.20 g, 10.6 mmol) dissolved in anhydrous diethyl ether (10 ml) was cooled to −40 degrees C., and the previously prepared allyl-magnesium bromide solution was added dropwise in such a manner that the reaction solution temperature was not elevated. After completion of the dropwise addition followed by stirring for 2 hours at −40 degrees C., slow heating was made up to 0 degrees C. After cooling with ice/water, the reaction solution was combined with a saturated ammonium chloride solution, and extracted with diethyl ether. The organic layer was washed with saturated sodium bicarbonate, water, and saturated brine, and then dried over anhydrous sodium sulfate. The solvent was distilled away under reduced pressure to obtain the desired substance.

Product: 1.06 g

Yield: 62%

Description: Pale yellow oil

NMR delta_{H} (400 MHz, CDCl₃):

2.31-2.42 (m, 4H), 3.49 (s, 2H), 5.15-5.21 (m, 4H), 5.79-5.90 (m, 2H).

### (2) Synthesis of intermediate 2,2-bis(2,2-dichlorocyclopropylmethyl)oxirane (Compound (II), R¹=2,2-dichlorocyclopropylmethyl, R²=2,2-dichlorocyclopropylmethyl)

4-chloromethylhepta-1,6-dien-4-ol (1.06 g, 6.6 mmol) was dissolved in chloroform (11 ml) and combined with benzyltriethylammonium chloride (0.15 g, 0.66 mmol). A solution of sodium hydroxide (5.20 g, 130 mmol) dissolved in water (5 ml) was added, and vigorous stirring was conducted for 15 hours at 60 degrees C. After the reaction followed by extraction with chloroform, the organic layer was washed with water and saturated brine, and dried over anhydrous sodium sulfate. The solvent was distilled away under reduced pressure, and the desired substance was obtained as a diastereomer mixture.

Product: 1.24 g

Yield: 65%

Description: Tan oil

NMR delta_{H} (400 MHz, CDCl₃):

1.12-1.17 (m, 2H), 1.57-1.82 (m, 5H), 1.95-2.10 (m, 3H), 2.81 (s, 0.5H), 2.81 (d, J=4.2 Hz, 0.5H), 2.92 (s, 0.5H), 2.94 (d, J=4.2 Hz, 0.5H).

### (3) Synthesis of 1,3-bis(2,2-dichlorocyclopropyl)-2-(1H-1,2,4-triazol-1-ylmethyl)propan-2-ol (Compound No. 1-277)

Under nitrogen flow, 60% sodium hydride (0.12 g, 3.0 mmol) was washed with hexane and then suspended in anhydrous DMF (5.0 ml), and combined with 1H-1,2,4-triazole (Compound (III), M=H) (0.20 g, 2.9 mmol) under cooling with ice. After stirring for 30 minutes at room temperature, a solution of 2,2-bis(2,2-dichlorocyclopropylmethyl)oxirane (Compound (II), R¹=2,2-dichlorocyclopropylmethyl, R²=2,2-dichlorocyclopropylmethyl) (0.58 g, 2.0 mmol) in anhydrous DMF (3.0 ml) was added, and stirring was conducted for 8 hours at 90 degrees C. The reaction solution was poured into ice/water, and extracted with ethyl acetate. The organic layer was washed with water and saturated brine, and then dried over anhydrous sodium sulfate. The solvent was distilled away under reduced pressure, and the resultant crude product was purified by silica gel column chromatography (eluent (hexane-ethyl acetate=2:1)) to obtain the desired substance.

<Compound No. I-277a>

Product: 82 mg

Yield: 11%

Description: White crystal, Melting point: 114 to 115 degrees C.

NMR delta_{H} (400 MHz, CDCl₃):

1.30 (t, J=7.4 Hz, 2H), 1.69-1.74 (m, 4H), 1.84-1.85 (m, 4H), 3.98 (s, 1H), 4.44 (s, 2H), 8.04 (s, 1H), 8.18 (s, 1H).

<Compound No. I-277b>

Product: 0.19 g

Yield: 26%

Description: White crystal, Melting point: 105 to 106.5 degrees C.

NMR delta_{H} (400 MHz, CDCl₃):

1.03-1.07 (m, 1H), 1.17-1.21 (m, 1H), 1.46-1.56 (m, 1H), 1.64-1.68 (m, 1H), 1.72-1.81 (m, 4H), 1.95-1.99 (m, 1H), 2.08 (dd, J=4.1, 14.8 Hz, 1H), 4.01 (d, J=1.4 Hz, 1H), 4.39 (d, J=14.1 Hz, 1H), 4.45 (d, J=14.1 Hz, 1H), 8.03 (s, 1H), 8.17 (s, 1H).

### Production Example 4

### Synthesis of 2-(1-chlorocyclopropyl)-4-(2,2-dichlorocyclopropyl)-1-(1H-1,2,4-triazol-1-yl)butan-2-ol (Compound No. I-607)

### (1) Synthesis of intermediate methyl 3-(1-chlorocyclopropyl)-3-oxopropionate (Compound (XIII), R²=1-chlorocyclopropyl, R¹³=Me)

Under nitrogen flow, 60% sodium hydride (3.80 g, 95.0 mmol) was washed with hexane and then suspended in dimethyl carbonate (Compound (XVI), R¹³=Me) (80 ml), combined with anhydrous methanol (0.5 ml) and warmed to 80 degrees C. A solution of 1-(1-chlorocyclopropyl)ethanone (Compound (XV), R²=1-chlorocyclopropyl) (10.2 g, 86.0 mmol) dissolved in dimethyl carbonate (Compound (XVI), R¹³=Me) (6 ml) was added, and stirring was conducted for 3 hours at 80 degrees C. After allowing to cool, the reaction solution was combined with acetic acid (10 ml), then poured into ice/water, and then the organic layer was fractionated. The aqueous layer was extracted with diethyl ether, and the each organic layer was washed with water and saturated brine, and then dried over anhydrous sodium sulfate. The solvent was distilled away under reduced pressure, and then distillation under reduced pressure gave the desired substance.

Product: 8.85 g

Yield: 58%

Description: Colorless oil, Boiling point: 88 degrees C.1.3 kPa

NMR delta_{H} (400 MHz, CDCl₃):

1.41 (d, J=5.1 Hz, 1H), 1.43 (d, J=4.8 Hz, 1H), 1.72 (d, J=4.8 Hz, 1H), 1.74 (d, J=5.1 Hz, 1H), 3.76 (s, 3H), 3.90 (s, 2H).

### (2) Synthesis of intermediate methyl 2-(2-propenyl)-3-(1-chlorocyclopropyl)-3-oxopropionate (Compound (XII), R²=1-chlorocyclopropyl, R¹⁴=H, R¹⁵=H, R¹⁶=H, R¹⁷=H, R¹⁸=H, R¹³=Me, m=1)

Under nitrogen flow, 60% sodium hydride (1.32 g, 33.0 mmol) was washed with hexane and then suspended in anhydrous DMF (70 ml). A solution of methyl 3-(1-chlorocyclopropyl)-3-oxopropionate (Compound (XIII), R²=1-chlorocyclopropyl, R⁹=Me) (5.30 g, 30.0 mmol) dissolved in anhydrous DMF (15 ml) was added, and stirring was conducted for 1.5 hours at room temperature. After stirring, a solution of allyl bromide (Compound (XIV), R¹⁴=H, R¹⁵=H, R¹⁶=H, R¹⁷=H, R¹⁸=H, X³=Br, m=1) (4.0 g, 33.0 mmol) dissolved in anhydrous DMF (15 ml) was added, and stirring was conducted for 3 hours at room temperature. The reaction solution was poured into ice/water, extracted with hexane, and the organic layer was washed with water and saturated brine, and dried over anhydrous sodium sulfate. The solvent was distilled away under reduced pressure to obtain the desired substance.

Product: 6.37 g

Yield: 98%

Description: Colorless oil

NMR delta_{H} (400 MHz, CDCl₃):

1.37-1.45 (m, 2H), 1.65-1.75 (m, 2H), 2.61-2.68 (m, 2H), 3.74 (s, 3H), 4.35 (t, J=7.0 Hz, 1H), 5.05-5.14 (m, 2H), 5.75-5.82 (m, 1H).

### (3) Synthesis of intermediate 1-(1-chlorocyclopropyl)-4-penten-1-on (Compound (XI), R²=1-chlorocyclopropyl, R¹⁴=H, R¹⁵=H, R¹⁶=H, R¹⁷=H, R¹⁸=H, m=1)

Methyl 2-(2-propenyl)-3-(1-chlorocyclopropyl)-3-oxopropionate (Compound (XII), R²=1-chlorocyclopropyl, R¹⁴=H, R¹⁵=H, R¹⁶=H, R¹⁷=H, R¹⁸=H, R¹³=Me, m=1) (6.16 g, 28.5 mmol) was dissolved in isopropanol (10 ml). A solution of sodium hydroxide (2.20 g, 55.0 mmol) dissolved in water (11 ml) was added, and stirring was conducted for 4.5 hours at 80 degrees C. After allowing to cool, the reaction solution was poured into ice/water, extracted with hexane, and the organic layer was washed with water and saturated brine, and dried over anhydrous sodium sulfate. The solvent was distilled away under reduced pressure, purification was made by silica gel column chromatography (eluent (hexane-ethyl acetate=50:1)) to obtain the desired substance.

Product: 3.0 g

Yield: 67%

Description: Colorless oil

NMR delta_{H} (400 MHz, CDCl₃):

1.31-1.35 (m, 2H), 1.62-1.65 (m, 2H), 2.31-2.37 (m, 2H), 2.94-2.98 (m, 2H), 4.98-5.09 (m, 2H), 5.78-5.87 (m, 1H).

### (4) Synthesis of intermediate 2-(3-butenyl)-2-(1-chlorocyclopropyl)oxirane (Compound (VIII-a), R²=1-chlorocyclopropyl, R¹⁴=H, R¹⁵=H, R¹⁶=H, R¹⁷=H, R¹⁸=H, m=1)

Under nitrogen flow, 60% sodium hydride (1.75 g, 43.7 mmol) was washed with hexane and then suspended in anhydrous DMSO (70 ml). Trimethylsulfoxonium bromide (7.51 g, 43.4 mmol) was added and stirring was conducted for 1.5 hours at room temperature. A solution of 1-(1-chlorocyclopropyl)-4-penten-1-on (Compound (IX), R²=1-chlorocyclopropyl, R¹⁴=H, R¹⁵=H, R¹⁶=H, R¹⁷=H, R¹⁸=H, m=1) (5.0 g, 31.5 mmol) dissolved in anhydrous DMSO (30 ml) was added, and stirring was further conducted for 3 hours at room temperature. The reaction solution was poured into ice/water, extracted with hexane, and the organic layer was washed with water and saturated brine, and dried over anhydrous sodium sulfate. The solvent was distilled away under reduced pressure to obtain an oil, which was distilled under reduced pressure to obtain the desired substance.

Product: 1.67 g

Yield: 31%

Description: Colorless oil

NMR delta_{H} (400 MHz, CDCl₃):

0.77-0.86 (m, 2H), 0.98-1.10 (m, 2H), 1.87-1.94 (m, 1H), 2.14-2.29 (m, 3H), 2.70 (d, J=4.9 Hz, 1H), 2.74 (d, J=4.9 Hz, 1H), 4.97-5.09 (m, 2H), 5.79-5.88 (m, 1H).

### (5) Synthesis of intermediate 2-(1-chlorocyclopropyl)-2-[2-(2,2-dichlorocyclopropyl)ethyl]oxirane (Compound (II-a), R²=1-chlorocyclopropyl, R⁸=H, R⁹=H, R¹⁰=H, R¹¹=H, R¹²=H, n=2)

2-(3-butenyl)-2-(1-chlorocyclopropyl)oxirane (Compound (VIII-a), R²=1-chlorocyclopropyl, R¹⁴=H, R¹⁵=H, R¹⁶=H, R¹⁷=H, R¹⁸=H, m=1) (18.92 g, 110 mmol) and benzyltriethylammonium chloride (515 mg, 2.26 mmol) were dissolved in chloroform (63 ml), combined with sodium hydroxide (23.07 g, 577 mmol)/water (23.5 ml), and stirred for 2 hours at 60 degrees C. The reaction solution was poured into ice/water, and extracted with chloroform. The organic layer was washed with water and saturated brine, and dried over anhydrous sodium sulfate. The solvent was distilled away under reduced pressure to obtain a crude product.

These procedures were repeated once more, and the resultant crude product was purified by distillation under reduced pressure, and the residue was purified by silica gel column chromatography (eluent (hexane-ethyl acetate=20:1)) to obtain the desired substance.

Product: 19.75 g

Yield: 71%

Description: Yellow oil

<Isomer a>

NMR delta_{H} (400 MHz, CDCl₃):

0.77-0.90 (m, 2H), 0.97-1.03 (m, 1H), 1.05-1.12 (m, 2H), 1.56-1.68 (m, 4H), 2.02-2.10 (m, 1H), 2.22-2.29 (m, 1H), 2.71-2.76 (m, 2H).

<Isomer b>

0.77-0.90 (m, 2H), 0.97-1.03 (m, 1H), 1.05-1.12 (m, 2H), 1.56-1.68 (m, 3H), 1.74-1.83 (m, 1H), 1.86-1.93 (m, 1H), 2.35-2.43 (m, 1H), 2.73 (d, J=4.9 Hz, 1H), 2.75 (d, J=4.9 Hz, 1H).

### (6) Synthesis of 2-(1-chlorocyclopropyl)-4-(2,2-dichlorocyclopropyl)-1-(1H-1,2,4-triazol-1-yl)butan-2-ol (Compound No. I-607)

Under nitrogen flow, 1H-1,2,4-triazole (Compound (III), M=H) (142 mg, 2.06 mmol), potassium carbonate (271 mg, 1.96 mmol), and potassium t-butoxide (15 mg, 0.13 mmol) were suspended in DMF (2 ml). A solution of 2-(1-chlorocyclopropyl)-2-[2-(2,2-dichlorocyclopropyl)ethyl]oxirane (Compound (II-a), R²=1-chlorocyclopropyl, R⁸=H, R⁹=H, R¹⁰=H, R¹¹=H, R¹²=H, n=2) (394 mg, 1.54 mmol) in DMF (2 ml) was added, and stirring was conducted for 5 hours at 70 degrees C. The reaction solution was poured into ice/water, and extracted with ethyl acetate. The organic layer was washed with water and saturated brine, and then dried over anhydrous sodium sulfate. The solvent was distilled away under reduced pressure, and the resultant crude product was purified crudely by silica gel column chromatography (eluent (hexane-ethyl acetate=1:1)), and then recrystallized from an ethyl acetate-hexane system to obtain the desired substance as 2 diastereomers.

<Compound No. I-607a>

Product: 40 mg

Yield: 8%

Description: White crystal, Melting point: 130 to 131 degrees C.

NMR delta_{H} (400 MHz, CDCl₃):

0.24 (ddd, J=11.0, 7.2, 6.0 Hz, 1H), 0.45 (ddd, J=10.7, 7.5, 6.0 Hz, 1H), 0.83 (ddd, J=10.7, 7.2, 5.7 Hz, 1H), 1.06 (ddd, J=11.0, 7.5, 5.7 Hz, 1H), 1.12 (bs, 1H), 1.5-1.6 (m, 2H), 1.6-1.8 (m, 1H), 1.8-2.1 (m, 3H), 4.06 (s, 1H), 4.27 (d, J=14.2 Hz, 1H), 4.71 (d, J=14.2 Hz, 1H), 8.01 (s, 1H), 8.24 (s, 1H).

<Compound No. I-607b>

Product: 55 mg

Yield: 11%

Description: White crystal, Melting point: 83 to 84 degrees C.

NMR delta_{H} (400 MHz, CDCl₃):

0.24 (ddd, J=11.0, 7.2, 6.0 Hz, 1H), 0.44 (ddd, J=10.8, 7.5, 6.0 Hz, 1H), 0.85 (ddd, J=10.8, 7.2, 5.7 Hz, 1H), 1.07 (ddd, J=11.0, 7.5, 5.7 Hz, 1H), 1.1-1.2 (m, 1H), 1.5-1.6 (m, 2H), 1.7-1.8 (m, 2H), 1.8-2.0 (m, 1H), 2.2-2.3 (m, 1H), 4.08 (s, 1H), 4.26 (d, J=14.2 Hz, 1H), 4.71 (d, J=14.2 Hz, 1H), 8.02 (s, 1H), 8.24 (s, 1H).

By the methods analogous to Production Example 1 to 4 described above, the following Compounds (I) shown in Table 38 to Table 41 below were synthesized.

TABLE 38

| Compound No. | Description | ¹H-NMR(CDCl₃) 400 MHz, δ |
|---|---|---|
| I-2b | Pale yellow | 0.31-0.37 (m, 1H), 0.43-0.49 (m, 1H), 0.74-0.79 (m, 1H), |
| | oil | 0.93-0.99 (m, 1H), 1.71 (dd, J = 11.0, 6.8 Hz, 1H), 1.83 (dd, J = 8.5, |
| | | 6.8 Hz, 1H), 2.35 (dd, J = 8.5, 0.9 Hz, 1H), 4.08 (s, 1H), 4.76 (d, J = |
| | | 14.2 Hz, 1H), 5.08 (d, J = 14.2 Hz, 1H), 8.01 (s, 1H), 8.31 (s, 1H). |
| I-12a | White solid | 0.53-0.66(m, 2H). 0.81-0.90 (m, 1H), 0.98-1.07(m, 1H), |
| | Melting | 1,45(s, 6H), 1.84(s, 1H), 4.02(s, 1H), 4.74 (d, J = 14.2 Hz, 1H), |
| | point: | 4,84(d, J = 14.2 Hz, 1H), 8.02(s, 1H), 8.30 (s, 1H). |
| | 86.4° C. | |
| I-12b | White solid | 0.72-0.79(m, 2H), 1.04-1.12(m, 1H), 1.25(s, 3H), 1.33-1.41(m, |
| | Melting | 1H), 1.38(s, 3H), 1.83(s, 1H), 3.66(s, 1H), 4.46 (d, |
| | point: | J = 14.2 Hz, 1H), 4.62(d, J = 14.2 Hz, 1H), 8.00 (s, 1H), 8.23 (s, 1H). |
| | 86.4° C. | |
| I-50a | Colorless oil | 1.18 (dd, J = 8.7, 7.5 Hz, 1H), 1.2-1.4 (m, 2H), 1.63 (dd, J = 10.8, |
| | | 8.9 Hz, 1H), 1.81 (dd, J = 10.5, 7.0 Hz, 1H), 2.0-2.1 (m, 2H), 2.32 |
| | | (d, J = 10.5 Hz, 1H), 3.32 (s, 1H), 4.48 (s, 2H), 7.97 (s, 1H), 8.19 |
| | | (s, 1H). |
| I-50b | White solid | 1.31 (dd, J = 8.9, 7.5 Hz, 1H), 1.35 (dd, J = 10.8, 7.5 Hz, 1H), 1.36 |
| | Melting | (t, J = 7.0 Hz, 1H), 1.73 (dd, J = 10.8, 8.9 Hz, 1H), 1.8-2.0 (m, |
| | point: | 2H), 2.02 (dd, J = 14.6, 7.5 Hz, 1H), 2.33 (dd, J = 14.6, 5.6 Hz, 1H), |
| | 129 to | 2.99 (s, 1H), 4.41 (s, 2H), 7.96 (s, 1H), 8.18 (s, 1H) |
| | 130° C. | |
| I-169a | Pale yellow | 1.16-1.22 (m, 1H), 1.29 (dd, J = 8.9, 7.4 Hz, 1H), 1.35 (dd, |
| | solid | J = 10.7, 7.3 Hz, 1H), 1.62-1.71 (m, 3H), 1.79-1.92 (m, 2H), 2.06-2.15 |
| | Melting | (m, 1H), 2.09 (dd, J = 9.8, 7.3 Hz, 1H), 2.73 (s, 1H), 4.28 (d, |
| | point: | J = 14.1 Hz, 1H), 4.33 (d, J = 14.1 Hz, 1H), 7.94 (s, 1H), 8.15 (s, 1H). |
| | 97.1° C. | |
| I-169b | Pale yellow | 1.16-1.21 (m, 1H), 1.35 (dd, J = 9.0, 7.3 Hz, 1H), 1.39 (dd, |
| | solid | J = 10.5, 7.3 Hz, 1H), 1.56-1.80 (m, 4H), 1.84-1.99 (m, 2H), 2.09-2.22 |
| | Melting | (m, 1H), 2.69 (brs, 1H), 4.27 (d, J = 14.1 Hz, 1H), 4.33(d, |
| | point: | J = 14.1 Hz, 1H), 7.94 (s, 1H), 8.16 (s, 1H). |
| | 79.8° C. | |

TABLE 39

| Compound No. | Description | ¹H-NMR(CDCl₃) 400 MHz, δ |
|---|---|---|
| I-192a | White solid | 0.32 (ddd, J = 13.2, 7.2, 6.0 Hz, 1H), 0.48 (ddd, J = 10.7, 7.5, 6.0 Hz, |
| | Melting | 1H), 0.81(ddd, J = 10.7, 7.2, 5,7 Hz, 1H), 1.07 (ddd, J = 10.7, 7.5, |
| | point: | 5.7 Hz, 1H), 1.2-1.3 (m, 1H), 1.72 (dd, J = 10.7, 7.2 Hz, 1H), 1.9-2.1 |
| | 73 to 74° C. | (m, 1H), 2.1-2.2 (m, 2H), 4.34 (bs, 1H), 4.37 (d, J = 14.2 Hz, 1H), |
| | | 4.90 (d, J = 14.2 Hz, 1H), 8.04 (s, 1H), 8.27 (s, 1H). |
| I-192b | White solid | 0.29 (ddd, J = 11.0, 7.2, 6.0 Hz, 1H), 0.46 (ddd, J = 10.7, 7.5, 6.0 Hz, |
| | Melting | 1H), 0.88(ddd, J = 10.7, 7.2, 5.7 Hz, 1H), 1.12 (ddd, J = 11.0, 7.5, |
| | point: | 5.7 Hz, 1H), 1.2-1.3 (m, 1H), 1.7-1.8 (m, 2H), 1.9-2.0 (m, 1H), 2.46 |
| | 102 to | (dd, J = 14.5, 4.8 Hz, 1H), 4.27 (bs, 1H), 4.32 (d, J = 14.2 Hz, 1H), |
| | 103° C. | 4.77 (d, J = 14.2 Hz, 1H), 8.03(s, 1H), 8.26 (s, 1H). |
| I-195 | Yellow | 0.81-0.86(m, 2H), 1.05-1.14(m, 1H), 1.18(d, J = 7.0 Hz, 3H), |
| (Isomer mixture) | gum | 1.20-1.30(m, 2H), 1.41-1.47(m, 1H), 1.60-1.65(m, 1H), |
| | | 1.84-1.89(m, 1H), 4.48(d, J = 14.8 Hz, 1H), 4.74(d, J = 14.8 Hz, 1H), |
| | | 7.91(s, 1H), 8.12(s, 1H). |
| I-198a | Pale yellow | 0.71-0.84(m, 2H), 0.91-0.98 (m, 1H), 0.99-1.07 (m, 1H), 1.37 |
| | solid | (d, J = 7.3 Hz, 1H), 1.39 (s, 3H), 1.54 (d, 1H, J = 7.3 Hz), 2.15 (s, 2H), |
| | | 4.63 (d, J = 15.0 Hz, 1H), 4.68 (d, J = 15.0 Hz, 1H), 7.92 (s, 1H), 8.17 |
| | | (s, 1H). |
| I-210b | White solid | 0.25-0.35 (m, 1H), 0.42-0.52 (m, 1H), 0.83-0.94 (m, 1H), |
| | Melting | 1.07- 1.17 (m, 1H), 1.38 (app. t, J = 7.5 Hz, 1H), 1.73 (dd, J = 8.7, |
| | point: | 14.5 Hz, 1H), 1.87 (dd, J = 7.1, 10.3 Hz, 1H), 1.93-2.07 (1H, m), |
| | 103° C. | 2.49 (dd, J = 4.4, 14.5 Hz, 1H), 4.27 (1H, s), 4.32 (d, J = 14.1 Hz, |
| | | 1H), 4.77 (d, J = 14.1 Hz, 1H), 8.03 (s, 1H), 8.25 (s, 1H). |

TABLE 40

| Compound No. | Description | ¹H-NMR(CDCl₃) 400 MHz, δ |
|---|---|---|
| I-616a | White solid | 0.20-0.27 (m, 1H), 0.41-0.47(m, 1H), 0.81-0.86 (m, 1H), 1.02-1.08 |
| | Melting | (m, 1H), 1.14-1.18 (m, 1H), 1.21 (s, 3H), 1.36 (s, 3H), 1.64-1.97 |
| | point: | (m, 4H), 4.04 (s, 1H), 4.27 (d, J = 14.1 Hz, 1H), 4.71 (d, |
| | 132 to | J = 14.1 Hz, 1H), 8.02 (s, 1H), 8.24 (s, 1H). |
| | 133° C. | |
| I-616b | White solid | 0.20-0.26 (m, 1H), 0.41-0.47 (m, 1H), 0.81-0.87 (m, 1H), 1.03-1.09 |
| | Melting | (m, 1H), 1.14-1.18 (m, 1H), 1.20 (s, 3H), 1.36 (s, 3H), 1.65-2.15 |
| | point: | (m, 4H), 4.06 (s, 1H), 4.25 (d, J = 14.1 Hz, 1H), 4.70 (d, |
| | 99 to 100° C. | J = 14.1 Hz, 1H), 8.02 (s, 1H), 8.24 (s, 1H). |
| I-625a | White solid | 0.23-0.28 (m, 1H), 0.42-0.48 (m, 1H), 0.80-0.86 (m, 1H), |
| | Melting | 1.03-1.07 (m, 1H), 1.26-1.29 (m, 1H), 1.61-1.81 (m, 3H), |
| | point: | 1.84-1.92 (m, 1H), 2.00-2.26(m, 2H), 4.05(s × 2, 1H), 4.28 (d, |
| | 134 to 135° C. | J = 14.1 Hz, 1H), 4.72 (d, J = 14.1 Hz, 1H), 8.02 (s, 1H), 8.25 (s, 1H). |
| I-625b | White solid | 0.23-0.28 (m, 1H), 0.42-0.48(m, 1H), 0.80-0.86 (m, 1H), 1.03-1.07 |
| | Melting | (m, 1H), 1.26-1.29 (m, 1H), 1.61-1.81 (m, 3H), 1.84-1.92 |
| | point: | (m, 1H), 2.00-2.26 (m, 2H), 4.09 (s × 2, 1H), 4.26 (d, |
| | 83 to 84° C. | J = 14.1 Hz, 1H), 4.72 (d, J = 14.1 Hz, 1H), 8.02 (s, 1H), 8.25 (s, 1H). |

TABLE 41

| Compound No. | Description | ¹H-NMR(CDCl₃) 400 MHz, δ |
|---|---|---|
| I-637a | Colorless | 0.56(m, 1H), 0.71(m, 1H), 0.90(m, 1H), |
| | solid | 1.06~1.14(m, 2H) 1.52-1.64(m, 2H), |
| | Melting | 1.69~1.93(m, 3H), 2.13(td, 1H, |
| | point | J = 12.7, 3.8 Hz), 3.26(br, 1H), |
| | 170 to | 4.09(d, 1H, J = 14.4 Hz), 4.24(d, |
| | 171° C. | 1H, J = 14.4 Hz), 7.01(s, 1H), |
| | | 7.03(s, 1H), 7.53(s, 1H). |

Respective intermediates used as described above can be synthesized also by the following Reference Production Examples.

### Reference Production Example 1

### Synthesis of intermediate 2-(1-chlorocyclopropyl)-2-(2,2-dibromocyclopropylmethyl)oxirane (Compound (II-a), R²=1-chlorocyclopropyl, R⁸=H, R⁹=H, R¹⁰=H, R¹¹=H, R¹²=H, X¹=Br, X²=Br, n=1)

### (1) Synthesis of intermediate 1-chloro-2-(1-chlorocyclopropyl)-4-penten-2-ol (Compound (IX), R²=1-chlorocyclopropyl, R⁸=H, R⁹=H, R¹⁰=H, R¹¹=H, R¹²=H, X=Cl, n=1)

To a mixture of 2-chloro-1-(1-chlorocyclopropyl)ethanone (30.6 g, 0.20 mol) combined with allyl bromide (36.3 g, 0.20×1.5 mol), THF (100 ml) and saturated aqueous ammonium chloride (200 ml), zinc (5.0 g, 0.020×1.15 mol) was added in 3 portions at an interval of 10 minutes, and then zinc (4.5 g, 0.20×0.38 mol) was added 10 minutes later. This reaction was conducted at 35 degrees C. or below. Since the starting material was found to be remaining after stirring for 2 hours, allyl bromide (3.63 g, 0.20×0.15 mol) and zinc (1.95 g, 0.020×0.15 mol) were added and stirring was conducted for 0.5 hour. The reaction solution was combined with concentrated hydrochloric acid (20 ml) and the organic layer was separated and used in the next reaction.

### (2) Synthesis of intermediate 2-(1-chlorocyclopropyl)-2-(2-propenyl)oxirane (Compound (VIII), R²=chlorocyclopropyl, R⁸=H, R⁹=H, R¹⁰=H, R¹¹=H, R¹²=H, n=1)

The organic layer obtained in (1) was combined with a 12.5% NaOH aqueous solution (128 g, 0.20×2.0 mol), and after stirring for 3 hours at room temperature further combined with a 25% NaOH aqueous solution (6.4 g, 0.20×0.2 mol) and stirred for 1 hour. The reaction solution was combined with hexane (100 ml), partitioned, and the aqueous layer was extracted with hexane (200 ml). The resultant organic layer was dried over anhydrous sodium sulfate, concentrated to obtain a crude product, which was distilled under reduced pressure to obtain the desired substance.

Product: 27.37 g

Yield: 88%

Description: Colorless oil

NMR delta_{H} (400 MHz, CDCl₃): 0.80 (ddd, J=10.8, 7.5, 5.4 Hz, 1H), 0.91 (ddd, J=10.3, 7.5, 5.4 Hz, 1H), 1.0-1.2 (m, 2H), 2.64 (ddt, J=14.9, 7.6, 1.1 Hz, 1H), 2.69 (d, J=5.1 Hz, 1H), 2.74 (d, J=5.1 Hz, 1H), 2.81 (ddt, J=14.9, 6.8, 1.1 Hz, 1H), 5.11 (ddt, J=10.2, 1.9, 1.1 Hz, 1H), 5.17 (ddt, J=17.2, 1.9, 1.4 Hz, 1H), 5.7-5.9 (m, 1H).

### (3) Synthesis of intermediate 2-(1-chlorocyclopropyl)-2-(2,2-dibromocyclopropylmethyl)oxirane (Compound (II-a), R²=1-chlorocyclopropyl, R⁴=H, R⁵=H, R⁶=H, R⁷=H, R⁸=H, X¹=Br, X²=Br, n=1)

2-(1-chlorocyclopropyl)-2-(2-propenyl)oxirane (Compound (VIII), R²=chlorocyclopropyl, R⁸=H, R⁹=H, R¹⁰=H, R¹¹=H, R¹²=H, n=1) (5.00 g, 31.5 mmol) and benzyltrimethylammonium chloride (0.29 g, 1.56 mmol) were dissolved in a mixed solution of bromoform (7.0 ml) and dichloromethane (7.0 ml). Under heating at about 60 degrees C., a 50% aqueous solution of sodium hydroxide (25.2 g, 0.31 mol) was added, and reaction was conducted for 15 hours. After allowing to cool to room temperature, the reaction solution was poured to ice/water, and extracted with dichloromethane. The organic layer was washed with water and saturated brine, and dried over anhydrous sodium sulfate. The resultant crude product was purified by silica gel column chromatography (eluent (hexane-ethyl acetate=50:1)) to obtain the desired substance.

Product: 8.97 g

Yield: 86%

Description: Pale yellow oil

<Isomer a>

NMR delta_{H} (400 MHz, CDCl₃):

0.83-0.89 (m, 1H), 0.93-0.99 (m, 1H), 1.02-1.12 (m, 2H), 1.36-1.40 (m, 1H), 1.58-1.66 (m, 1H), 1.79-1.84 (m, 1H), 2.17 (dd, 1H, J=8.0, 15.4 Hz), 2.34 (dd, 1H, J=5.9, 15.4 Hz), 2.83 (d, 1H, J=4.7 Hz), 2.97 (d, 1H, J=4.7 Hz).

<Isomer b>

NMR delta_{H} (400 MHz, CDCl₃):

0.84-0.90 (m, 1H), 0.93-0.99 (m, 1H), 1.06-1.15 (m, 2H), 1.32-1.36 (m, 1H), 1.73-1.82 (m, 2H), 1.86-1.92 (m, 1H), 2.53-2.58 (m, 1H), 2.77 (d, 1H, J=4.8 Hz), 2.85 (d, 1H, J=4.8 Hz).

### Reference Production Example 2

### Synthesis of intermediate methyl 2-(2-propenyl)-3-(1-chlorocyclopropyl)-3-oxopropionate (Compound (XII), R²=1-chlorocyclopropyl, R¹⁴=H, R¹⁵=H, R¹⁶=H, R¹⁷=H, R¹⁸=H, R¹³=Me, m=1)

Under nitrogen flow, dimethyl carbonate (Compound (XVI), R¹³=Me) (80 ml) and 1-(1-chlorocyclopropyl)ethanone (Compound (XV), R²=1-chlorocyclopropyl) (10.2 g, 86.0 mmol) were heated to 75 degrees C. A 28% methanol solution of sodium methoxide (7.0 ml, 33.7 mmol) was added, and while taking methanol out of the system the 28% methanol solution of sodium methoxide (7.0 ml, 33.7 mmol) was further added 3 times at an interval of 30 minutes, and thereafter heating was conducted for 3.5 hours with stirring.

After reducing the reaction temperature to 60 degrees C., a solution mixture of allyl bromide (Compound (XIV), R¹⁴=H, R¹⁵=H, R¹⁶=H, R¹⁷=H, R¹⁸=H, X³=Br, m=1) (7.85 ml, 90.7 mmol) and dimethyl carbonate (Compound (XVI), R⁹=Me) (20 ml) was added dropwise. Heating was conducted for 1.5 hours with stirring. The reaction solution was poured into ice/water and extracted with hexane. The organic layer was washed with water and saturated brine, and dried over anhydrous sodium sulfate. The solvent was distilled away and the resultant oil was purified by silica gel column chromatography (eluent (hexane-ethyl acetate=30:1)) to obtain the desired substance.

Product: 12.87 g

Yield: 69%

### Reference Production Example 3

### Synthesis of intermediate 2-(1-chlorocyclopropyl)-2-[2-(2,2-dichlorocyclopropyl)ethyl]oxirane (Compound (II-a), R¹=1-chlorocyclopropyl, R⁸=H, R⁹=H, R¹⁰=H, R¹¹=H, R¹²=H, n=2)

### (1) Synthesis of intermediate methyl 2-(2-propenyl)-3-(1-chlorocyclopropyl)-3-oxopropionate (Compound (XII), R¹=1-chlorocyclopropyl, R¹⁴=H, R¹⁵=H, R¹⁶=H, R¹⁷=H, R¹⁸=H, R¹³=Me, m=1)

Under nitrogen flow, 60% sodium hydride (11.13 g, 0.253 mol) was washed with hexane and then suspended in dimethyl carbonate (220 ml), to which anhydrous methanol (1.5 ml, 0.253×0.146 mol) was added and warming was conducted to 80 degrees C. A solution of 1-(1-chlorocyclopropyl)ethanone (Compound (XV), R²=1-chlorocyclopropyl) (30 g, 0.253 mol) in dimethyl carbonate (20 ml) was added in portions. After completion of the addition, stirring was conducted for 4 hours at 80 degrees C. After adjusting the reaction temperature at 60 degrees C., allyl bromide (Compound (XIV), R¹⁴=H, R¹⁵=H, R¹⁶=H, R¹⁷=H, R¹⁸=H, X³=Br, m=1) (33.67 g, 0.253×1.1 mol) was added dropwise and the reaction was conducted for 3 hours. The reaction was further conducted for 1 hour at 65 degrees C. The reaction solution was allowed to cool to room temperature, and then poured into ice/water, and the organic layer was extracted with hexane (100 ml×3 times). The organic layer was washed with water, dried over anhydrous sodium sulfate, concentrated to obtain the desired substance as a crude material, which was used in the next reaction.

Crude product: 58.9 g

### (2) Synthesis of intermediate 1-(1-chlorocyclopropyl)-4-penten-1-on (Compound (XI), R²=1-chlorocyclopropyl, R¹⁴=H, R¹⁵=H, R¹⁶=H, R¹⁷=H, R¹⁸=H, m=1)

Methyl 2-(2-propenyl)-3-(1-chlorocyclopropyl)-3-oxopropionate (Compound (XII), R²=1-chlorocyclopropyl, R¹⁴=H, R¹⁵=H, R¹⁶=H, R¹⁷=H, R¹⁸=H, R¹³=Me, m=1) obtained as a crude material in (1) (27.0 g, 0.125 mol) was dissolved in methanol (125 ml). A 2N aqueous solution of sodium hydroxide (9.97 g, 0.1246×2 mol being dissolved in 125 ml of water) was added dropwise in such a manner that the internal temperature was kept below room temperature (about 22 to 23 degrees C.). Thereafter, stirring was conducted for about 3 hours and 20 minutes at room temperature. Then, the reaction solution was treated with 20 ml of acetic acid to adjust the pH at about 5, and then heated to about 80 degrees C., and stirred for about 30 minutes with heating.

The reaction solution was allowed to cool to room temperature, and extracted with diethyl ether (100 ml×3). The organic layer was washed with water, and then washed with saturated brine. The organic layer was concentrated, and the residue was distilled under reduced pressure (b.p.: 61 to 64/1.4 kPa) to obtain the desired substance.

Product: 14.0 g

Yield: 76% (Overall yield from 1-(1-chlorocyclopropyl)ethanone (Compound (XV), R²=1-chlorocyclopropyl))

### (3) Synthesis of intermediate 2-(1-chlorocyclopropyl)-2-(3-butenyl)oxirane

(Compound (VIII-a), R²=1-chlorocyclopropyl, R¹⁴=H, R¹⁵=H, R¹⁶=H, R¹⁷=H, R¹⁸=H, m=1)

1-(1-chlorocyclopropyl)-4-penten-1-on (Compound (IX), R²=1-chlorocyclopropyl, R¹⁴=H, R¹⁵=H, R¹⁶=H, R¹⁷=H, R¹⁸=H, m=1) (5.00 g, 31.5 mmol), trimethylsulfoxonium bromide (7.64 g, 44.1 mmol) and diethylene glycol (0.2 ml, 2.11 mmol) were suspended in toluene (25 ml). Under heating at about 75 degrees C., a ground solid 85% potassium hydroxide (2.48 g, 37.6 mmol) was added, and the reaction was conducted for 1 hour. After allowing to cool to room temperature, insolubles were filtered and washed with hexane (50 ml). The filtrate was washed with water and saturated brine, and dried over anhydrous sodium sulfate. The solvent was distilled away to obtain the desired substance.

Product: 5.06 g

Yield: 93%

### (4) Synthesis of intermediate 2-(1-chlorocyclopropyl)-2-[2-(2,2-dichlorocyclopropyl)ethyl]oxirane (Compound (II-a), R¹=1-chlorocyclopropyl, R⁸=H, R⁹=H, R¹⁰=H, R¹¹=H, R¹²=H, n=2)

2-(1-chlorocyclopropyl)-2-(3-butenyl)oxirane (Compound (VIII-a), R²=chlorocyclopropyl, R¹⁴=H, R¹⁵=H, R¹⁶=H, R¹⁷=H, R¹⁸=H, m=2) (26.0 g, 150 mmol) and benzyltriethylammonium chloride (1.71 g, 7.54 mmol) were dissolved in chloroform (90 ml). A 50% aqueous solution of sodium hydroxide (120 g, 1.50 mol) was added, and the reaction was conducted for 5 hours under heating at about 50 degrees C. After allowing to cool to room temperature, the chloroform layer was fractionated, and the aqueous layer was extracted further with hexane. The organic layer was washed with water and saturated brine, and dried over anhydrous sodium sulfate. The organic layer was concentrated, and then distilled under reduced pressure to obtain the desired substance.

Product: 33.9 g

Yield: 88%

Description: Colorless oil, Boiling point: 101 degrees C.0.13 kPa

The following Compounds (II) were synthesized by the methods analogous to the abovementioned Production Examples 1 to 4 and Reference Production Examples 1 to 3.

TABLE 42

| Compound | | |
|---|---|---|
| No. | R1 | R2 |
| II-12 | 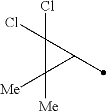 | 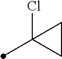 |
| II-50 | 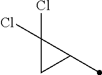 | 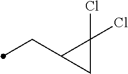 |
| II-169 | 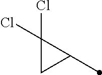 | 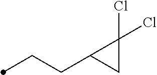 |
| II-192 | 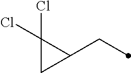 | 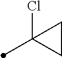 |
| II-195 | 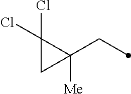 | 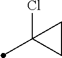 |
| II-616 | 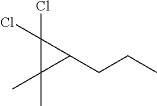 | 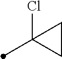 |
| II-625 | 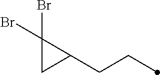 | 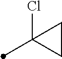 |

TABLE 43

| Compound No. | Description | ¹H-NMR(CDCl₃) 400 MHz, δ |
|---|---|---|
| II-12ab | Colorless oil | 0.79-1.15(m, 4H), 1.27(d, J = 7.3 Hz, 0.5H), 1.29(d, J = 8.0 Hz, 0.5H), 1.37(s, 1.5H), |
| (Mixture of | | 1.44(s, 1.5H), 1.52(d, J = 7.3 Hz, 0.5H), 1.63(d, J = 7.3 Hz, 0.5H), 2.08(d, J = 15.4 Hz, |
| two isomers) | | 0.5H), 2.24(d, J = 15.3 Hz, 0.5H), , 2.33(d, J = 15.2 Hz, 0.5H), 2.55(d, J = 15.4 Hz, |
| | | 0.5H), 2.77(d, J = 4.9 Hz, 1H), 2.82(d, J = 4.9 Hz, 0.5H), 2.84(d, J = 4.9 Hz, 0.5H). |
| II-50a | Yellow oil | 1.20 (t, J = 6.0 Hz, 1H), 1.38 (dd, J = 8.0, 6.8 Hz, 1H), 1.47 (dd, J = 10.5, |
| | | 6.8 Hz, 1H), 1.6-1.8 (m, 2H), 2.14 (dd, J = 10.5, 8.0 Hz, 1H), 2.20 (dd, J = |
| | | 15.3, 8.6 Hz, 1H), 2.34 (dd, J = 15.3, 4.9 Hz, 1H), 2.52 (d, J = 4.8 Hz, 1H), |
| | | 2.85 (d, J = 4.8 Hz, 1H). |
| II-50b | Yellow oil | 1.27 (dt, J = 14.9, 6.8 Hz, 1H), 1.41 (dd, J = 8.0, 6.8 Hz, 1H), 1.49 (dd, J = |
| | | 10.7, 6.8 Hz, 1H), 1.7-1.9 (m, 2H), 2.12 (dd, J = 14.9, 5.9 Hz, 1H), 2.21 (dd, J = |
| | | 10.7, 8.0 Hz, 1H), 2.21 (dd, J = 14.6, 7.6 Hz, 1H), 2.52 (dd, J = 5.1, 0.8 Hz, |
| | | 1H), 2.78 (d, J = 5.1 Hz, 1H). |
| II-169a | Pale yellow oil | 1.09-1.16(m, 1H), 1.36(dd, J = 8.0, 6.3 Hz, 1H), 1.46(dd, J = 10.7, 8.6 Hz, 1H), 1.58-1.67(m, |
| | | 2H), 1.71-1.82(m, 2H), 1.94(ddd, J = 13.6, 9.2, 6.7 Hz, 1H), 2.12(dd, J = 10.7, |
| | | 8.1 Hz, 1H), 2.23-2.31(m, 1H), 2.473and2.475(d, J = 5.0 Hz, 1H), 2.69(d, J = 5.0 |
| | | Hz, 1H). |
| II-169b | Pale yellow oil | 1.10-1.18(m, 1H), 1.37(dd, J = 8.0, 6.8 Hz, 1H), 1.46(dd, J = 10.6, 6.8 Hz, 1H), 1.59-1.85(m, |
| | | 4H), 1.86-1.95(m, 1H), 2.12(dd, J = 10.6, 8.0 Hz, 1H), 2.35-2.44(m, 1H), 2.45(d, |
| | | J = 5.0 Hz, 1H), 2.68(d, J = 5.0 Hz, 1H). |
| II-192a | Pale yellow oil | 0.83-0.90(m, 1H), 0.93-0.98(m, 1H), 1.02-1.16(m, 2H), 1.20-1.28(m, |
| | | 1H), 1.61-1.68(m. 2H), 2.14-2.20(m. 1H), 2.33-2.38(m, 1H), |
| | | 2.81(d, 1H, J = 4.7 Hz), 2.94(d, 1H, J = 4.7 Hz). |
| II-192b | Pale yellow oil | 0.83-0.90(m, 1H), 0.93-0.98(m, 1H), 1.02-1.16(m, 2H), 1.17-1.21(m, 1H), 1.63-1.66(m, |
| | | 2H), 1.91 (dd, 1H, J = 7.8, 7.3 Hz), 2.53(dd, 1H, J = 6.1, 8.9 Hz), |
| | | 2.76(d, 1H, J = 4.9 Hz), 2.83(d, 1H, J = 4.9 Hz). |
| II-195a | Colorless oil | 0.79-0.85(m, 1H), 0.91-0.98(m, 1H), 1.01-1.09(m, 2H), 1.27(d, J = 7.3 Hz, 1H), 1.37(s, |
| | | 3H), 1.63(d, J = 7.2 Hz, 1H), 2.08(d, J = 15.4 Hz, 1H), 2.55(d, J = 15.4 Hz, 1H), 2.77(d, |
| | | J = 4.9 Hz, 1H), 2.84(d, J = 4.9 Hz, 1H). |
| II-195b | Pale yellow oil | 0.79-1.15(m, 4H), 1.29(d, J = 8.0 Hz, 1H), 1.44(s, 3H), 1.52(d, J = 7.3 Hz, 1H), 2.24(d, |
| | | J = 15.3 Hz, 1H), 2.33(d, J = 15.3 Hz, 1H), 2.77(d, J = 4.9 Hz, 1H), 2.82(d, J = |
| | | 4.9 Hz, 1H). |
| II-616ab | Pale yellow oil | 0.78-0.91 (m, 2H), 0.97-1.11(m, 2H), 1.14-1.17(m, 1H), 1.18(s, 3H), |
| | (Isomer mitxture) | 1.35(s, 3H), 1.53-1.68(m, 2H), 1.82-2.00(m, 1H), 2.15-2.32(m, 1H), |
| | | 2.71-2.76(m, 2H) |
| II-625a | Pale yellow oil | 0.78-0.92(m, 2H), 0.99-1.04(m, 1H), 1.06-1.12(m, 1H), 1.22-1.29(m, 1H), |
| | | 1.55-1.61(m, 2H), 1.63-1.82(m, 2H), 2.04-2.13(m, 1H), 2.22-2.30(m, 1H), |
| | | 2.72-2.77(m, 2H). |
| II-625b | Pale yellow oil | 0.78-0.92(m, 2H), 0.99-1.04(m, 1H), 1.06-1.12(m, 1H), 1.22-1.29(m, 1H), |
| | | 1.55-1.61(m, 2H), 1.63-1.82(m, 2H), 1.88-1.95(m, 1H), 2.49-2.58(m, 1H), |
| | | 2.72-2.77(m, 2H). |

The followings are Formulation Examples and Experimental Examples. Carriers (diluents) and auxiliary agents, as well as the mixing ratio thereof for active ingredients may vary within a wide range. “Parts” in each Formulation Example means “parts by weight”.

### Formulation Example 1

### Wettable Formulation

Compound (1-192a) 50 parts

Lignin sulfonate 5 parts

Alkyl sulfonate 3 parts

Diatomaceous earth 42 parts

were ground and mixed to form a wettable formulation, which was used as being diluted in water.

### Formulation Example 2

### Powder Formulation

Compound (I-607a) 3 parts

Clay 40 parts

Talc 57 parts

were ground and mixed, and used as a dusting formulation.

### Formulation Example 3

### Granule Formulation

Compound (I-625a) 5 parts

Bentonite 43 parts

Clay 45 parts

Lignin sulfonate 7 parts

were mixed uniformly, combined with water and further kneaded, and subjected to an extruding granulator to obtain a granule, which was dried and used as a granule formulation.

### Formulation Example 4

### Emulsion Formulation

Compound (1-210a) 20 parts

Polyoxyethylene alkylaryl ether 10 parts

Polyoxyethylene sorbitan monolaurate 3 parts

Xylene 67 parts

were mixed and dissolved uniformly to obtain an emulsion formulation.

### Experimental Example 1

### Efficacy Test Against Cucumber Gray Mold

Onto a cucumber (variety:SHARP1) plant in its cotyledon phase grown using a square plastic pot (6 cm×6 cm) to cultivate, a wettable formulations such as Formulation Example 1 which was diluted and suspended in water at a certain concentration (500 mg/L) was sprayed at a rate of 1,000 L/ha. The sprayed leaves were air-dried, and loaded with a paper disc (8 mm in diameter) soaked in a spore suspension of Botrytis cinerea, and kept at 20 degrees C. and a high humidity. Four days after inoculation, the cucumber gray mold lesion degree was investigated, and the protective value was calculated by the following equation.

Protective value(%)=(1−mean lesion degree in sprayed plot/mean lesion degree in unsprayed plot)×100

TABLE 44

| Lesion degree | % Area of onset |
|---|---|
| 0 | No onset |
| 0.5 | % Area of lesion spot < 10% |
| 1 | 10% ≦ % Area of lesion spot < 20% |
| 2 | 20% ≦ % Area of lesion spot < 40% |
| 3 | 40% ≦ % Area of lesion spot < 60% |
| 4 | 60% ≦ % Area of lesion spot < 80% |
| 5 | 80% ≦ % Area of lesion spot |

In the test described above, Compounds I-2a, I-2b, I-192a, I-192b, I-210a, I-607a, I-607b, I-625a, I-625b, for example, showed protective values of 80% or higher.

### Experimental Example 2

### Efficacy Test Against Wheat Brown Rust

Onto a wheat plant (variety:NORIN No. 61) grown to the two-leaf phase using a square plastic pot (6 cm×6 cm), a wettable formulations such as Formulation Example 1 which was diluted and suspended in water at a certain concentration (500 mg/L) was sprayed at a rate of 1,000 L/ha. The sprayed leaves were air-dried, and inoculated with spore suspension of Puccinia recondita (adjusted at 200 spores/vision, Gramin S was added at 60 ppm) by spraying, and kept at 25 degrees C. and a high humidity for 48 hours. Thereafter, the plant was kept in a greenhouse. Nine to fourteen days after inoculation, the wheat brown rust lesion degree was investigated, and the protective value was calculated by the following equation.

Protective value(%)=(1−mean lesion degree in sprayed plot/mean lesion degree in unsprayed plot)×100

TABLE 45

| Lesion degree | Leaf rust damage scale by Peterson |
|---|---|
| 0 | No onset |
| 0.5 | Less than 1% |
| 1 | 1% or higher and less than 5% |
| 2 | 5% or higher and less than 10% |
| 3 | 10% or higher and less than 30% |
| 4 | 30% or higher and less than 50% |
| 5 | 50% or higher |

In the test described above, Compounds I-2a, I-2b, I-50a, I-192a, I-192b, I-210a, I-210b, I-277a, I-277b, I-607a, I-607b, I-625a, I-625b, for example, showed protective values of 80% or higher.

### Experimental Example 3

### Efficacy Test Against Wheat Fusarium Head Blight

Onto a head of a wheat plant (variety:NORIN No. 61) grown to the blooming phase, a wettable formulations such as Formulation Example 1 which was diluted and suspended in water at a certain concentration (500 mg/L) was sprayed at a rate of 1,000 L/ha. The head was air-dried, and inoculated with spore suspension of Fusarium graminearum (adjusted to 2×10⁵ spores/ml, containing Gramin S at a final concentration of 60 ppm and sucrose at a final concentration of 0.5%) by spraying, and kept at 20 degrees C. and a high humidity. Four to seven days after inoculation, the wheat fusarium head blight lesion degree was investigated, and the protective value was calculated by the following equation.

Protective value(%)=(1−mean lesion degree in sprayed plot/mean lesion degree in unsprayed plot)×100

TABLE 46

| Lesion degree | % Area of onset |
|---|---|
| 0 | No onset |
| 0.2 | Less than 1% |
| 0.5 | 1% or higher and less than 3% |
| 1 | 3% or higher and less than 5% |
| 2 | 5% or higher and less than 10% |
| 3 | 10% or higher and less than 25% |
| 4 | 25% or higher and less than 50% |
| 5 | 50% or higher |

In the test described above, Compounds I-2a, I-2b, I-192a, I-192b, I-210a, I-210b, I-607a, I-607b, I-625a, I-625b, for example, showed protective values of 80% or higher.

### Experimental Example 4

### Assay for Fungicidal Effect on Various Pathogenic Microorganism and Hazardous Microorganisms

In this Experimental Example, the fungicidal effects on various phytopathogenic fungi for plants and hazardous microorganism for industrial materials were examined.

Compound (I) was dissolved in 2 ml of dimethyl sulfoxide. 0.6 ml of this solution was added to 60 ml of a PDA medium (potato dextrose agar medium) and at about 60 degrees C., which was mixed thoroughly in a 100-ml conical flask, and poured into a dish, where it was solidified, thereby obtaining a plate medium containing the inventive compound at a certain concentration.

On the other hand, a subject microorganism previously cultured on a plate medium was cut out using a cork borer whose diameter was 4 mm, and inoculated to the test compound-containing plate medium described above. After inoculation, the dish was grown at the optimum growth temperatures for respective microorganisms (for this growth temperature, see, for example, LIST OF CULTURES 1996 microorganisms 10th edition, Institute for Fermentation (foundation)) for 1 to 3 days, and the mycelial growth was measured as a diameter of its flora. The growth degree of the microorganism on the test compound-containing plate medium was compared with the growth degree of the microorganism in the untreated group, and % mycelial growth inhibition was calculated by the following equation.

R=100(dc−dt)/dc

wherein R=% mycelial extension inhibition, dc=flora diameter in untreated plate, dt=flora diameter in treated plate.

The results obtained were evaluated as one of the 5 grades in accordance with the following criteria.

<Growth Inhibition Grade>

5: % Mycerial growth inhibition of 80% or higher

4: % Mycerial growth inhibition of less than 80 to 60% or higher

3: % Mycerial growth inhibition of less than 60 to 40% or higher

2: % Mycerial growth inhibition of less than 40 to 20% or higher

1: % Mycerial growth inhibition of less than 20%

TABLE 47

| | Concentration | | | | | | | | | | | |
|---|---|---|---|---|---|---|---|---|---|---|---|---|
| Compound | mg/ml | P.n | P.h | F.g | U.n | P.o | G.f | A.m | S.s | B.c | F.c | R.sec |
| I-2a | 50 | 5 | 5 | 5 | 5 | 5 | 5 | 5 | 5 | 5 | 5 | 5 |
| I-12a | 50 | 5 | 5 | 5 | 5 | 5 | 5 | 5 | 5 | 5 | 5 | 5 |
| I-50a | 50 | 5 | 5 | 5 | 5 | 5 | 5 | 5 | 5 | 5 | 5 | 5 |
| I-50b | 50 | 5 | 5 | 5 | 5 | 5 | 5 | 5 | 5 | 5 | 5 | 5 |
| I-192a | 50 | 5 | 5 | 5 | 5 | 5 | 5 | 5 | 5 | 5 | 5 | 5 |
| I-192b | 50 | 5 | 5 | 5 | 5 | 5 | 5 | 5 | 5 | 5 | 5 | 5 |
| I-210a | 50 | 5 | 5 | 5 | 5 | 5 | 5 | 5 | 5 | 5 | 5 | 5 |
| I-210b | 50 | 5 | 5 | 5 | 5 | 5 | 5 | 5 | 5 | 5 | 5 | 5 |
| I-607a | 50 | 5 | 5 | 5 | 5 | 5 | 5 | 5 | 5 | 5 | 5 | 5 |
| I-607b | 50 | 5 | 5 | 5 | 5 | 5 | 5 | 5 | 5 | 5 | 5 | 5 |
| I-625a | 50 | 5 | 5 | 5 | 5 | 5 | 5 | 5 | 5 | 5 | 5 | 5 |
| I-625b | 50 | 5 | 5 | 5 | 5 | 5 | 5 | 5 | 5 | 5 | 5 | 5 |

Wheat Septoria nodorum blotch microorganism (Phaeosphaeria nodorum) P.n
Wheat eye spot (Pseudocercoporella herpotrichoides) P.h
Wheat fusarium blight (Fusarium graminearum) F.g
Barley loose smut (Ustilago nuda) U.n
Rice blast (Pyricularia oryzae) P.o
Rice bakanae disease (Giberella fujikuroi) G.f
Alternaria blotch (Alternaria alternata) A.m
Sclerotinia rot (Sclerotinia sclerotiorum) S.s
Gray mold (Botritis cinerea) B .c
Cucumber fusarium wilt (Fusarium oxysporum) F.c
Barley leaf blotch (Rhynchosporium secalis) R.sec

Also in the experiments with the treatment at 50 mg/l against a microorganism which deteriorates paper, pulp, fiber, leather, paint and the like, namely, Aspergillus microorganism (Aspergillus sp.), Tricoderma microorganism (Trichoderma sp.), Penicillium microorganism (Penicillium sp.), Cladosporium microorganism (Cladosporium sp.), Mucor microorganism (Mucor sp.), Aureobasidium microorganism (Aureobasidium sp.), Curvularia microorganism (Curvularia sp.), a wood denaturing microorganism Oouzuratake (Tyromyces palustris) and Kawaratake, (Coriolus versicolor), Compounds I-2a, I-12a, I-50a, I-50b, I-192a, I-192b, I-210a, I-210b, I-607a, I-607b, I-625a, I-625b showed growth inhibition grades of as high as 4 or more.

### Experimental Example 5

### Wheat Elongation Prevention Assay

2 mg of a test compound was dissolved in 18 micro litre of DMSO, and applied to 1 g of wheat seeds in a vial. One day later, the seeds were seeded to 1/10000 a pots at a rate of 10 seeds/pot, and then cultivated in a greenhouse with supplying water underneath. Fourteen days after seeding, the plant height of the seedlings in each treatment group was surveyed in 10 locations, and the % plant height suppression was calculated by the following Equation.

R=1000(hc−ht)/hc

wherein R=% plant height suppression, hc=mean untreated plant height, ht=mean treated plant height.

The results obtained were assigned to one of the following 5 grades of the growth regulation in accordance with the following criteria.

### <Growth Regulation Grade>

5: % Plant height suppression of 50% or higher

4: % Plant height suppression of less than 50 to 30% or higher

3: % Plant height suppression of less than 30 to 20% or higher

2: % Plant height suppression of less than 20 to 10% or higher

1: % Plant height suppression of less than 10%

In the assay described above, Compounds I-2a, I-192a, I-192b, I-210a, I-210b, I-607a, I-607b, I-625a, I-625b showed growth regulation grades of 4 or higher in the growth of rice plant.

### INDUSTRIAL APPLICABILITY

An azole derivative according to the invention can preferably be utilized as an active ingredient of agro-horticultural bactericides, plant growth regulators and industrial material protecting agents.

1. An azole derivative represented by Formula (I): 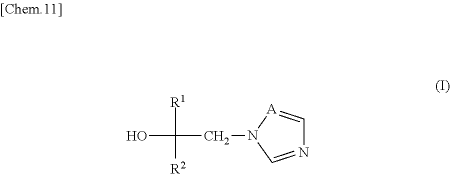
wherein R¹ and R² are same or different, and each denotes a C3-C6 cycloalkyl group or a C1-C4 alkyl group substituted with the group;
the cycloalkyl group and the alkyl group may be substituted with a halogen atom, a C1-C4 alkyl group, a C1-C4 haloalkyl group, a C3-C6 cycloalkyl group, an aryl group or an arylalkyl group (alkyl moiety carbon chain being C1-C3);
the aromatic ring of the aryl group and the arylalkyl group may be substituted with a halogen atom, a C1-C4 alkyl group, a C1-C4 haloalkyl group, a C1-C4 alkoxy group or a C1-C4 haloalkoxy group; and,
A denotes a nitrogen atom or a methyne group.

2. The azole derivative according to claim 1 wherein each of R¹ and R² in Formula (I) described above is a C3-C6 cycloalkyl group substituted with a halogen atom, a C1-C4 alkyl group, or a C1-C4 haloalkyl group, or,
a C1-C4 alkyl group substituted with the substituted C3-C6 cycloalkyl group.

3. The azole derivative according to claim 1 wherein each of R¹ and R² in Formula (I) described above is a cyclopropyl group substituted with a halogen atom or a C1-C4 alkyl group, or,
a C1-C4 alkyl group substituted with the substituted cyclopropyl group.

4. The azole derivative according to claim 1 wherein each of R¹ and R² in Formula (I) described above is represented by Formula (XVII): 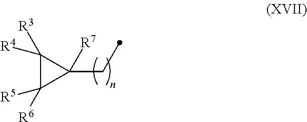
wherein each of R³, R⁴, R⁵, R⁶, and R⁷ denotes a hydrogen atom, a halogen atom, a methyl group, or an ethyl group, and at least one of R³, R⁴, R⁵, R⁶, and R⁷ denotes a halogen atom, and n denotes 0 to 2.

5. The azole derivative according to claim 4 wherein, when n in Formula (XVII) described above representing R¹ in Formula (I) described above is 1 to 2, then n in Formula (XVII) described above representing R² in Formula (I) described above is 0 while R⁷ is a halogen atom and each of R³, R⁴, R⁵, and R⁶ is a hydrogen atom.

6. The azole derivative according to claim 1 wherein A in Formula (I) described above is a nitrogen atom.

7. An intermediate compound for the azole derivative according to claim 1 represented by Formula (II): 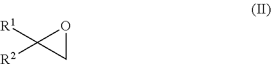
wherein R¹ and R² are same or different, and each denotes a C3-C6 cycloalkyl group, a C1-C4 alkyl group substituted with the cycloalkyl group, a C2 alkenyl group, or a C1-C4 alkyl group substituted with the alkenyl group; the cycloalkyl group, the alkyl group, or the alkenyl group may be substituted with a halogen atom, a C1-C4 alkyl group, a C1-C4 haloalkyl group, a C3-C6 cycloalkyl group, an aryl group, or an arylalkyl group (alkyl moiety carbon chain being C1-C3); the aromatic ring of the aryl group and the arylalkyl group may be substituted with a halogen atom, a C1-C4 alkyl group, a C1-C4 haloalkyl group, a C1-C4 alkoxy group, or a C1-C4 haloalkoxy group.

8. The intermediate compound according to claim 7 represented by Formula (II-a): 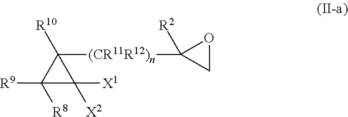
wherein R⁸, R⁹, R¹⁰, R¹¹, and R¹² may be substituted with a hydrogen atom, a halogen atom, a C1-C4 alkyl group, a C1-C4 haloalkyl group, a C3-C6 cycloalkyl group, an aryl group, or an arylalkyl group (alkyl moiety carbon chain being C1-C3); the aromatic ring of the aryl group and the arylalkyl group may be substituted with a halogen atom, a C1-C4 alkyl group, a C1-C4 haloalkyl group, a C1-C4 alkoxy group, or a C1-C4 haloalkoxy group;
each of X¹ and X² denotes a halogen atom; and,
n denotes 0 to 4.

9. The intermediate compound according to claim 7 represented by Formula (VIII): 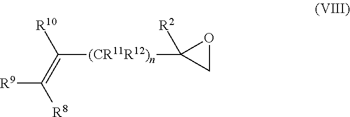
wherein each of R⁸, R⁹, R¹⁰, R¹¹, and R¹² denotes a hydrogen atom, a halogen atom, a C1-C4, alkyl group, a C1-C4 haloalkyl group, a C3-C6 cycloalkyl group, an aryl group or an arylalkyl group (alkyl moiety carbon chain being C1-C3); the aromatic ring of the aryl group and the arylalkyl group may be substituted with a halogen atom, a C1-C4 alkyl group, a C1-C4 haloalkyl group, a C1-C4 alkoxy group, or a C1-C4 halo alkoxy group; and,
n denotes 0 to 4.

10. A method for producing the azole derivative according to claim 1 comprising a step of reacting an oxirane compound represented by Formula (II): 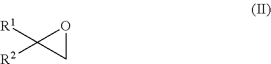
with a 1,2,4-triazole or imidazole compound represented by Formula (III): 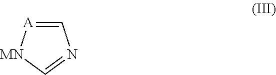
wherein M denotes a hydrogen atom or an alkaline metal; and A denotes a nitrogen atom or a methyne group.

11. A method for producing an intermediate compound for producing the intermediate compound according to claim 8 comprising a step of subjecting an oxirane compound represented by Formula (VIII) to conversion into a gem-dihalocyclopropane thereby obtaining an intermediate compound represented by Formula (II-a).

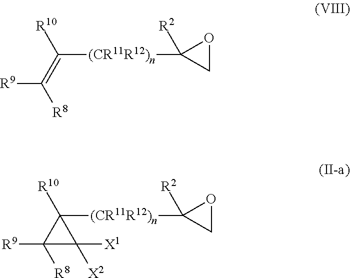

12. A method for producing an intermediate compound for producing the intermediate compound according to claim 9 comprising a step of allowing a compound represented by Formula (VII) to react with an organometallic compound represented by Formula (X), to obtain a halohydrin compound represented by Formula (IX) which is then subjected to conversion into an oxirane, thereby obtaining an intermediate compound represented by Formula (VIII): 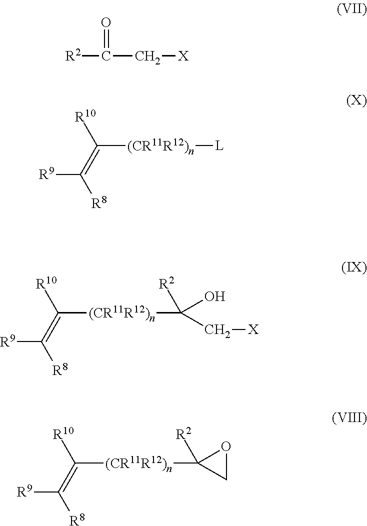
wherein L in Formula (X) denotes an alkaline metal, an alkaline earth metal-Q₁ (Q₁ is an halogen atom), a ½ (Cu alkaline metal), a zinc-Q₂ (Q₂ is a halogen atom), and X in Formulae (VII) and (IX) denotes a halogen atom.

13. A method for producing an intermediate compound for producing the intermediate compound according to claim 9 comprising a step of subjecting a carbonyl compound represented by Formula (XI) to conversion into an oxirane thereby obtaining an intermediate compound represented by Formula (VIII-a): 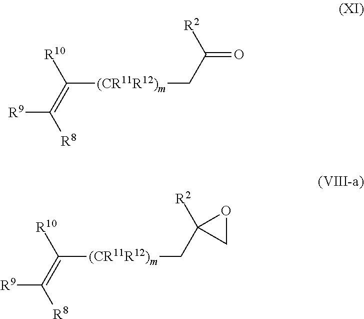
wherein m in Formula (XI) and (VIII-a) denotes 1 to 3.

14. An agro-horticultural agent or an industrial material protecting agent containing as an active ingredient the azole derivative according to claim 1.
